(19) United States
(12) Patent Application Publication
(10) Pub. No.: US 2018/0256233 A1
(43) Pub. Date: Sep. 13, 2018

(54) TWO-PART STORAGE AND MIXING DEVICE FOR THE PRODUCTION OF A BONE CEMENT, AND PERTINENT METHOD

(71) Applicant: Heraeus Medical GmbH, Wehrheim (DE)

(72) Inventors: Sebastian Vogt, Erfurt (DE); Thomas Kluge, Vallendar (DE)

(21) Appl. No.: 15/914,194

(22) Filed: Mar. 7, 2018

(30) Foreign Application Priority Data
Mar. 8, 2017 (DE) 102017104854.5

(51) Int. Cl. A61B 17/88; B01F 3/12; B01F 15/00; B01F 15/02; B01F 13/00; B01F 5/06

(52) U.S. Cl. CPC A61B 17/8833; B01F 3/12; B01F 15/0087; B01F 15/0205; B01F 15/0237; B01F 15/0293; A61B 2017/8838; B01F 15/0278; B01F 5/0602; A61B 17/8827; A61B 17/8822; B01F 2003/1257; B01F 2215/0029; B01F 13/0023

(57) ABSTRACT

A storage and mixing device for the production of a bone cement dough from a monomer liquid and a cement powder as starting components of the bone cement. The storage and mixing device includes: (1) a receptacle having a monomer liquid container which contains the monomer liquid, (2) a cartridge containing the cement powder, (3) a feed plunger, which is accessible from a rear side of the receptacle, arranged in the receptacle, and movable in a longitudinal direction of the receptacle, and (4) a dispensing plunger which is arranged in the cartridge and shiftable in a longitudinal direction inside of the cartridge. The front side of the receptacle is connected or connectable to the rear side of the cartridge with a first securing element on the rear side of the receptacle and a second securing element on the rear side of the cartridge.

The invention also relates to a method for the production of a bone cement dough, in particular of a pasty polymethylmethacrylate bone cement dough, whereby the bone cement dough is produced from a cement powder and a monomer liquid through the use of a storage and mixing device.

### PRIORITY

This application claims priority under 35 U.S.C. § 119(a) to DE 10 2017 104 854.5, filed on Mar. 8, 2017, the entirety of which is incorporated herein by reference.

### TECHNICAL FIELD

The invention relates to a storage and mixing device for the production of a bone cement dough from a monomer liquid and a cement powder as starting components of the bone cement.

The invention also relates to a method for the production of a bone cement dough, in particular of a pasty polymethylmethacrylate bone cement dough, whereby the bone cement dough is produced from a cement powder and a monomer liquid through the use of a storage and mixing device.

The subject matter of the invention specifically is a device for separate storage of the cement powder and the monomer liquid of polymethylmethacrylate bone cements, for subsequent mixing of the cement powder with the monomer liquid in order to produce a bone cement dough, and for dispensing the mixed bone cement dough. The bone cement dough produced with the device is designed, in particular, for augmentation of fractured vertebral bodies, i.e. for vertebroplasty. The storage and mixing device according to the invention is a full-prepacked cementing system.

### BACKGROUND

Polymethylmethacrylate (PMMA) bone cements are based on the pioneering work of Sir Charnley (Charnley, J.: Anchorage of the femoral head prosthesis of the shaft of the femur. J. Bone Joint Surg. 42 (1960) 28-30.). Conventional polymethylmethacrylate bone cements (PMMA bone cements) are made from a powdered component and a liquid monomer component (K.-D. Kuhn: Knochenzemente fir die Endoprothetik: Ein aktueller Vergleich der physikalischen und chemischen Eigenschaften handelsiiblicher PMMA-Zemente. Springer-Verlag Berlin Heidelberg N.Y., 2001). The powder component, also called cement powder or bone cement powder, comprises one or more polymers that are produced through polymerisation, preferably suspension polymerisation, based on methylmethacrylate and co-monomers, such as styrene, methylacrylate or similar monomers, a radiopaquer, and the initiator, dibenzoylperoxide. Mixing the powder component and the monomer component, swelling of the polymers of the powder component in the methylmethacrylate generates a dough that can be shaped plastically and is the actual bone cement. During the mixing of powder component and monomer component, the activator, N,N-dimethyl-p-toluidine, reacts with dibenzoylperoxide while forming radicals. The radicals thus formed trigger the radical polymerisation of the methylmethacrylate. Upon advancing polymerisation of the methylmethacrylate, the viscosity of the cement dough increases until the cement dough solidifies.

A number of special polymethylmethacrylate bone cements have been developed for treatment of impression fractures of vertebral bodies. These are characterized in that they contain a relatively high fraction of radiopaquer, for example zirconium dioxide and barium sulphate. This is to facilitate continuous monitoring of the spreading of the bone cement dough in the fractured vertebra by means of fluoroscopy. Vertebroplasty and kyphoplasty are the processes for augmentation of fractured vertebral bodies used most commonly today. Currently, manual mixing of the cement components in mixing beakers or in simple mixing devices is common in this context.

PMMA bone cements can be mixed by mixing the cement powder and the monomer liquid in suitable mixing beakers with the aid of spatulas. This can lead to air bubbles being enclosed in the bone cement dough, which can have a negative effect on the mechanical properties of the cured bone cement.

A large number of vacuum cementing systems has been described for preventing air inclusions in bone cement dough of which the following shall be specified here for exemplary purposes: U.S. Pat. No. 6,033,105 A, U.S. Pat. No. 5,624,184 A, U.S. Pat. No. 4,671,263 A, U.S. Pat. No. 4,973,168 A, U.S. Pat. No. 5,100,241 A, WO 99/67015 A1, EP 1 020 167 A2, U.S. Pat. No. 5,586,821 A, EP 1 016 452 A2, DE 36 40 279 A1, WO 94/26403 A1, EP 1 005 901 A2, EP 1 886 647 A1, U.S. Pat. No. 5,344,232 A.

From EP 2 730 296 A2, a thixotropic bone cement for vertebroplasty is known, in which the thixotropic properties can be generated through the use of multiple additives.

Cementing systems, in which both the cement powder and the monomer liquid are already packed in separate compartments of the mixing devices and are mixed with each other in the cementing system only right before application of the cement, are a development of cementing technology. Said closed full-prepacked mixing devices have been proposed by means of EP 0 692 229 A1, DE 10 2009 031 178 B3, U.S. Pat. No. 5,997,544 A, U.S. Pat. No. 6,709,149 B1, DE 698 12 726 T2, EP 0 796 653 A2, and U.S. Pat. No. 5,588,745 A.

Patent DE 10 2009 031 178 B3 discloses a storage and mixing device as a full-prepacked mixing device, in which the starting components required for the production of the bone cement dough are stored already in the storage and mixing device and can be combined and mixed in the storage and mixing device. The storage and mixing device comprises a two-part dispensing plunger for closing a cement cartridge. A combination of a gas-permeable sterilization plunger and a gas-impermeable sealing plunger is used in this context. This principle of a closed vacuum mixing system is implemented in the closed cementing system, PALACOS® PRO, made and distributed by the company Heraeus Medical GmbH.

After mixing the cement powder with the liquid monomer component, polymethylmethacrylate bone cements are applied in their non-cured pasty state in the form of a cement dough. If mixing devices are used with powder-liquid cements, the cement dough is situated in a cartridge. The cement dough is squeezed from said cartridge through the motion of a dispensing plunger. The dispensing plungers usually have a diameter of between 30 mm and 40 mm and thus have a surface area of 7.0 cm² to 12.5 cm² on the outside that is engaged by the pestle of the extrusion device during the extrusion process. The motion of the dispensing plunger is effected by manually operated mechanical extrusion devices, which are also called applicators. Said manual extrusion devices usually reach an extrusion force in the range of approximately 1.5 kN to 3.5 kN.

The application of conventional PMMA bone cements, which consist of a liquid monomer component and a separately stored cement powder component as starting components, involves the two starting components being mixed in cementing systems and the cement dough thus formed then being extruded by means of manually operated extrusion devices. These simple mechanical extrusion devices utilize, in particular, clamp rods that are driven by a manually-actuated tilting lever for extrusion. The manually driven extrusion devices are time-proven throughout the world for decades and as such are the current prior art. Said extrusion devices are advantageous in that the medical user has a feel for the penetration resistance of the bone cement dough into the bone structures (cancellous bone) by means of the manual force to be expended.

The use of all full-prepacked mixing devices known to date requires the medical user to perform multiple working steps in a predetermined order on the devices one after the other until the mixed bone cement dough is ready and can be applied. Any confusion of the working steps can lead to failure of the mixing device and can therefore cause a disturbance in the surgical procedure. Cost-intensive training of the medical users is therefore required in order to prevent user errors from occurring.

WO 00/35506 A1 proposes a device, in which the polymethylmethacrylate cement powder is stored in a cartridge, whereby the cement powder takes up the entire volume of the cartridge and the volume of the intervening spaces between the particles of the cement powder is equal to the volume of the monomer liquid required for the production of bone cement dough using the cement powder stored in the cartridge. The design of said device is appropriate such that the action of a vacuum causes the monomer liquid to be supplied into the cartridge from above, whereby a vacuum is applied to a vacuum connector on the underside of the cartridge for this purpose. As a result, the monomer liquid is aspirated through the cement powder, whereby the air present in the intervening spaces of the cement powder particles is replaced by the monomer liquid. This involves no mechanical mixing of the cement dough thus formed by means of a stirrer.

It is a disadvantage of the system that cement powders, which swell quickly due to the monomer liquid, cannot be mixed with said device, because the rapidly swelling cement powder particles form a gel-like barrier of approximately 1 to 2 cm after ingress of the monomer liquid into the cement powder and impede the migration of the monomer liquid through the entire cement powder. Moreover, conventional cement powders show a phenomenon, which is that the cement powder particles are wetted only poorly by methylmethacrylate due to the difference in surface energies. As a result, the methylmethacrylate penetrates only slowly into the cement powder. Moreover, it cannot be excluded that the monomer liquid, exposed to the action of a vacuum, is aspirated through the vacuum connector after the monomer liquid fully penetrates into the cement powder. In this case, an insufficient amount of monomer liquid for curing by means of radical polymerisation is available and/or the mixing ratio and thus the consistency of the bone cement is changed inadvertently. Moreover, it is a problem that the air trapped between the cement powder particles is to be displaced by the monomer liquid proceeding from top to bottom, because the air, having a lower specific weight than the monomer liquid, tends to migrate upwards in the cement powder rather than downwards in the direction of the vacuum connector under the force of gravity.

In the case of high viscosity pasty bone cements used with cartridges, in which the dispensing plunger has a total surface area in the range of 7.0 cm² to 12.5 cm² at the external plunger side, which is engaged by a pestle of the extrusion devices, these devices can be operated manually either not at all or only while expending a very large force. This is true even more, if the flow resistance of the bone cement dough to be extruded is increased by an extended dispensing opening and/or by a static mixer, as is common in applications at the spine, in which the bone cement dough is dispensed via a hose or a trocar. This exertion of a large force is unreasonable for medical users in the OR.

From the adhesives and sealant industry, electrically driven extrusion devices are known as well. Said devices can be driven both with rechargeable batteries and batteries or by means of a stationary electrical power supply. Said devices can extrude particularly thick pasty masses since their extrusion force is very large in some cases. However, it is one disadvantage of the use of electrical motors that these motors contain non-ferrous metals and are expensive purchases. Since the OR area needs to be kept sterile, said devices need to be sterilized with much effort or may even need to be replaced. The presence of electrical wiring may impede the mobility of the user in the OR.

Moreover, pneumatic devices have been proposed as well. Said devices require a stationary or mobile compressed air connection (U.S. Pat. No. 2,446,501 A, DE 20 2005 010 206 U1). This necessitates compressed air hoses, which may impede the mobility of the user.

Alternatively, the use of compressed gas cartridges to provide compressed gas is feasible just as well. Devices have been proposed for this purpose, in which the supply of compressed gas is controlled by a valve and, in addition, the flow of the viscous mass is controlled by a second valve (US 2004/0074927 A1, U.S. Pat. No. 6,935,541 B1). In these devices, the gas cartridges are integrated into the devices. These systems, which are connected to compressed air or contain compressed gas cartridges, always necessitate the presence of a compressed gas source in the absence of which the systems cannot be used.

In vertebroplasty, the application of bone cement is monitored in situ by means of an x-ray procedure. Application devices for vertebroplasty usually have a hose inserted in them through the tip of which the bone cement can be applied to allow the user to work outside the range of the x-rays. For this purpose, a trocar or a cannula can be arranged as well on the hose. Said systems are known, for example, from U.S. Pat. No. 7,112,205 B2, U.S. Pat. No. 8,038,682 B2, U.S. Pat. No. 8,308,731 B2, DE 10 2005 045 227 A1, EP 1 074 231 B1, EP 1 596 736 B1, U.S. Pat. No. 9,005,209 B2, and WO 2008/097855 A2.

Alternatively, other set-ups can be used for keeping the user away from the x-rays, such as are described, for example, in documents U.S. Pat. No. 6,676,663 B2, U.S. Pat. No. 7,008,433 B2, U.S. Pat. No. 8,348,494 B2, EP1 464 292 B, EP 1 614 403 B, US 2008/319445 A9, and WO 2008/038322 A2.

A bone cement applicator for vertebroplasty for application of bone cement comprising a hose, a trocar, and a mixer is known from US 2008/0086143 A1. The bone cement applicator comprises two cartridges arranged next to each other, in which the starting components are stored as well. The bone cement applicator is assembled right before use. In bone cement applicators for vertebroplasty of this type, pressure is exerted on the starting components of the bone cement by means of an extrusion device propelling the dispensing plungers in the cartridges, and the pressure is used to expel the starting components from the cartridges and through the hose. In this context, the starting components are usually mixed first in an upstream static mixer. As a result, the parts of the bone cement applicator serving as borders to the bone cement flow (the cartridges, the housing of the mixer, and the hose) are subject to elastic deformation. When the propulsion of the dispensing plunger is stopped, the elastic force of said parts leads to a volume contraction of said parts such that bone cement continues to exit through the application opening of the hose and/or trocar. This may lead to contamination of the operation theatre or of the user with bone cement or an excessive amount of the bone cement is applied inadvertently. Moreover, when the volume flow of the bone cement dough is to be started up again, pressure needs to be established in the bone cement first to make the bone cement exit through the application opening. This, in turn, delays the time point after commencement of the propulsion of the dispensing plungers from which the bone cement can actually be applied, which is also undesirable. Since the bone cement dough and the starting components are highly viscous, in particular where pasty starting components are used, all these effects are relatively strongly pronounced. This can be counteracted by the use of massive and expensive metallic housing parts. Said parts need to be cleaned after use and need to be sterilized for further use or need to be recycled with much effort. Moreover, residual starting components may be released when the cartridges are taken off and may contaminate the operating room (OR).

U.S. Pat. No. 8,544,683 B2 discloses a cartridge system that is suitable for admixing a small amount to a main starting component. The cartridge system has, aside from a cartridge, a second smaller cartridge arranged in it, whereby, along with the propulsion of a dispensing plunger in the larger cartridge, a dispensing plunger in the smaller cartridge is also driven by a joint connecting element. However, the system is not suitable for mixing the viscous pasty starting components of PMMA bone cement.

Accordingly, it is the object of the invention to overcome the disadvantages of the prior art. Specifically, it is the object of the invention to develop a storage and mixing device and a method, which both are well-suited for mixing and subsequent dispensing and preferably also for storing a cement powder and a monomer liquid as starting components of a polymethylmethacrylate bone cement. Moreover, a method for the production of a prepacked PMMA cement cartridge system, which is well-suited for mixing and applying a PMMA bone cement dough and overcomes said problems, is to be provided. It shall be feasible to drive the storage and mixing device with a conventional extrusion device, and the storage and mixing device shall be operable as easy as possible. The design is to be inexpensive to allow the storage and mixing device to be used a single time only for hygienic reasons. As many as possible or all of the processes taking place in the storage and mixing device, such as the mixing of the starting components, the dispensing of the bone cement dough, and the opening of containers and, if applicable, of the cartridge, are to take place in the smallest possible number of working steps and are to be automated to the extent possible and preferably are to be driven by a single drive only.

Preferably, the invention is to also provide a simple and inexpensively manufactured bone cement applicator for vertebroplasty for pasty multicomponent polymethylmethacrylate bone cements and a method for the application of a cement dough with a storage and mixing device that has a simple design and is inexpensive to manufacture, whereby the bone cement dough does not continue to flow once the cement flow is stopped. Moreover, it shall be possible to use the storage and mixing device again as soon as possible after interruption of the flow of bone cement dough. Contamination of the surroundings and of the user with bone cement dough or the starting components, in particular the monomer liquid, shall be excluded to the extent possible.

Accordingly, it is the object of the invention, specifically, to develop a storage and mixing device for storing and mixing cement powder and monomer liquid, whereby the polymethylmethacrylate bone cement dough produced by mixing the starting components is preferably intended for the augmentation of fractured vertebral bodies. The handling of the storage and mixing device is to be maximally simplified in order to prevent, as a matter of rule, user errors resulting from assembly steps taking place incorrectly. The medical user is to be enabled to connect the storage and mixing device, after taking it out of a packaging, to an extrusion device and to subsequently manually actuate said facility. The storage and mixing device is to ensure the secure storage of cement powder and monomer liquid in separate compartments such that any inadvertent mixing of the cement components during storage of the storage and mixing device is excluded. The storage and mixing device is to allow for sterilization with ethylene oxide gas. The cement powder stored in the storage and mixing device must be accessible to ethylene oxide. It shall be feasible to activate the storage and mixing device with the aid of a manually driven extrusion device of the type currently common in the OR such that, after form-fitting or force-locked connection of the storage and mixing device or of parts thereof to the extrusion device, the pestle of the extrusion device acts on the storage and mixing device due to the extrusion device being actuated and opens the monomer liquid container and subsequently, if the punch moves further, transfers the monomer liquid into the cement powder. The mixing of the monomer liquid and the cement powder is to take place without the aid of a mixer that is to be moved manually from outside. Just the forward motion of the pestle of the extrusion device is to effect the opening of the monomer liquid container, the subsequent monomer transfer into the cement powder, and the mixing of the cement components while forming the cement dough. It is important to design the dispensing plunger for dispensing the cement dough thus formed appropriately such that the pressure acting on the dispensing plunger upon the use of a common manual extrusion device is sufficiently high such that the cement dough can be extruded through a plastic hose with an internal diameter of 3 mm over a distance of at least 20 cm.

It shall be possible to easily manufacture the storage and mixing device from plastics and thus the storage and mixing device shall be suitable as a product for single use. It shall be possible to extrude the mixed cement dough with a conventional manually-driven extrusion device of the type that is hitherto conventional for use with PMMA bone cements for the cementing of knee and hip TEP (total endoprosthesis of the hip joint). The bone cement applicator is to be designed appropriately such that an immediate emergency stop of the flowing bone cement dough is feasible without contamination of the surgical theatre (OR—operation room) by the bone cement dough and/or continued flow of bone cement dough taking place.

Preferably, the bone cement applicator to be developed shall not necessitate two pestles that are connected to each other and are propelled synchronously in order for the entire device not to become significantly more extensive, longer, and larger than the mixing devices that are thus far customary for the conventional powder-liquid polymethylmethacrylate bone cements. The aim is to find a simple solution that allows the bone cement dough to be extruded, if possible with just one pestle and, if applicable, a cup connected to it.

### SUMMARY OF THE INVENTION

It is therefore the object of the invention to develop a simple storage and mixing device and a method, for storing and mixing cement powder and monomer liquid that does not require any complicated devices, is reproducible regardless of the operating personnel, and rapidly provides a bone cement for application.

The objects of the invention are met by a storage and mixing device for the production of a bone cement dough from a monomer liquid and a cement powder as starting components of the bone cement, the storage and mixing device comprising
A) a receptacle, having a monomer liquid container containing the monomer liquid, and
B) a cartridge containing the cement powder,
C) a feed plunger contacting the receptacle, whereby the feed plunger is movable in a longitudinal direction of the receptacle and is accessible from a rear side of the receptacle, and
D) a dispensing plunger in the cartridge, whereby the dispensing plunger is movable in a longitudinal direction within the cartridge, whereby
E) a front side of the receptacle is connected or connectable to a rear side of the cartridge such that an internal space of the receptacle is in fluid communication with an internal space of the cartridge such as to be permeable to the monomer liquid, whereby the connection is detachable, and whereby
F) a first securing element is provided on the rear side of the receptacle and a second securing element is provided on the rear side of the cartridge, whereby the first securing element and the second securing element are identical or congeneric.

Preferably, the first securing element and the second securing element are identical. Specifically, both the receptacle and the cartridge are securable in the same manner on the same holder of an extrusion device, such as a cartridge gun, and therefore the receptacle and the cartridge can be extruded with the same extrusion device. The term cartridge gun shall be understood to also include caulking guns. It is preferred to use a manually-driven extrusion device or cartridge gun.

According to the invention, the storage and mixing device is preferred to have a two-part design.

The invention can therefore preferably provide the first securing element and the second securing element to be suitable and provided to be secured to a single holder of an extrusion device.

It is preferable to use a polymethylmethacrylate bone cement (PMMA bone cement) as bone cement. Accordingly, in one embodiment, the cement powder is a PMMA cement powder.

The invention can preferably provide the dispensing plunger to be accessible from a rear side of the cartridge when the receptacle and the cartridge are not connected to each other.

Preferably, the monomer liquid container is a container that is separate from the receptacle.

The invention can just as well provide a dispensing opening, in particular as part of a dispensing tube, to be provided on the front side of the cartridge for dispensation of the bone cement dough. In this context, the invention can preferably provide a gas-permeable closure, which can be taken off or is openable, to close the dispensing opening for cement powder particles. In this context, the invention can preferably provide that the closure is openable upon the application of axial pressure or the action of a manual force.

Storage and mixing devices can be provided, according to the invention, to have a closable feedthrough into the internal space of the cartridge on the rear side of the cartridge, in particular in the dispensing plunger of the cartridge, whereby the closable feedthrough preferably is closable in liquid tight manner upon the liquid-permeable connection between the receptacle and the cartridge being detached, whereby the closable feedthrough preferably comprises a valve, at least two sealing lips or a self-closing membrane that is puncturable.

The effect of designing the feedthrough to be closable is that, after feeding the monomer liquid into the cartridge, no monomer liquid can exit through same on the rear side of the cartridge after the receptacle is detached from the cartridge. As a result, contamination and/or soiling of the surgical area with the monomer liquid is prevented.

In this context, it is preferred for the invention to provide a filter, in particular a pore filter, that is impermeable to the cement powder and permeable to the monomer liquid to be arranged in the closable feedthrough. As a result, the cement powder can be prevented from penetrating into the closable feedthrough, from reacting there with the monomer liquid, from swelling there, and, from inadvertently closing the feedthrough.

Moreover, the invention can provide a hollow needle or a tube on the front side of the receptacle by which the inside of the receptacle is connectable to the internal space of the cartridge in liquid-conducting manner, whereby the hollow needle or the tube together with the closable feedthrough form the monomer liquid-permeable connection into the internal space of the cartridge, if the receptacle is connected to the cartridge.

What this embodiment attains is that the closure of the closable feedthrough is openable in simple and secure manner by using the hollow needle or the tube and that the closure can close again in self-acting manner, when the hollow needle or the tube are pulled out of the closable feedthrough. Obviously, multiple hollow needles or multiple tubes can be used for this purpose just as well.

The invention also proposes to have a connecting plunger arranged on the front side of the receptacle, and to have it arranged such as to be mobile in longitudinal direction in the inside of the receptacle, whereby the monomer liquid container is arranged between the connecting plunger and the feed plunger, whereby a passage is provided in the connecting plunger and the passage is permeable to the monomer liquid and gases and is usable to establish a fluid connection to the internal space of the cartridge, whereby, preferably, a motion of the connecting plunger in the direction of the cartridge a cartridge that is connected to the front side of the receptacle, the closable feedthrough on the rear side of the cartridge or a closing element that closes the liquid-permeable connection between the internal space of the cartridge and the internal space of the receptacle is openable.

What this embodiment attains is that the fluid connection between the internal space of the cartridge and the internal space of the receptacle is opened only when the connecting plunger is moved or that the storage and mixing device is easier and simpler in design. Due to the mobile connecting plunger, the internal space of the cartridge can be prevented from being opened inadvertently. Moreover, the motion of the connecting plunger can open the closable feedthrough such that the closure of the closable feedthrough is openable by the same linear drive that is also used to drive the feed plunger and the dispensing plunger.

Preferably, the hollow needle or the tube is arranged in the connecting plunger. In this context, the invention can preferably provide the connecting plunger, in the storage state of the storage and mixing device, to be arranged at an appropriate distance from the dispensing plunger or the cartridge in the receptacle such that the hollow needle or the tube does not touch the closure of the closable passage on the rear side of the cartridge. Whereby the hollow needle preferably does not touch a puncturable membrane of the closable passage or the tube does not touch the sealing lips or the valve of the closable passage. However, if the design involves sealing lips, the tube is preferably guided through the sealing lips already.

Moreover, the invention can provide, on the front side in the receptacle, a limit stop that limits the motion of the connecting plunger in the direction of the cartridge or the motion of the connecting plunger in the direction of the front side of the receptacle.

According to a preferred refinement, the invention can provide a screen or a porous disk that is permeable to gases and liquids on the front side of the receptacle in the liquid-permeable connection to the cartridge.

As a result, fragments or severed parts of the monomer liquid container can be prevented from getting into the cartridge and therefore into the bone cement. Moreover, said fragments or parts can also be prevented from impairing or blocking the liquid-permeable connection between the receptacle and the cartridge.

The invention can just as well provide the storage and mixing device to be a bone cement applicator for storage and mixing of the starting components and for application of the bone cement dough in the area of the spine, whereby the bone cement applicator preferably comprises an extended dispensing opening that is arranged at the front side of the cartridge, whereby the extended dispensing opening particularly preferably is implemented with a dispensing tube, a hose and/or a trocar.

The storage and mixing device can be used particularly well as bone cement applicator for vertebroplasty. Due to the linear two-part design and the ability to design the cartridge to be very narrow, it is feasible to utilize a manually-driven extrusion device although the bone cement dough is highly viscous and although the dispensing opening is long, possibly including trocar and hose, which increase the flow resistance even further. This is the case because the stroke distance with which the storage and mixing device, and thus the bone cement applicator, is driven is very long and, due to the elongated design and the ensuing relatively small internal diameter of the internal space of the cartridge, a long stroke will extrude only a small amount of the bone cement dough through the extended dispensing opening.

Preferably, the extended dispensing opening is implemented through a dispensing tube on which an adapter, in particular a Luer system adapter, for attachment of a hose or trocar is arranged. Preferably, the hose has an internal diameter of less than 4 mm. It is also preferred for the hose to have a minimum compressive strength of 10 bar.

Said storage and mixing devices that are intended to be bone cement applicators for spinal fusion can be provided appropriately such that a three-way valve that is operable from outside is arranged in the extended dispensing opening or between the extended dispensing opening and the cartridge, in that a collecting container for reception of bone cement dough is arranged on the three-way valve, whereby the extended dispensing opening merges into an application opening that is arranged on the end of the extended dispensing opening that faces away from the cartridge, whereby the three-way valve is designed appropriately such that it, being in a first position, provides a fluid connection between the application opening and the cartridge and closes a discharge channel toward the collecting container and, being in a second position, provides a fluid connection between the application opening and the collecting container and closes a passage toward the cartridge.

What this attains when the extrusion process is interrupted is that the pressure acting on the bone cement in an extended dispensing opening, such as a hose, can be removed by adjusting a three-way valve that is connected to the collecting container, the hose, and the cartridge, without any substantial amount of the bone cement dough continuing to flow. Simultaneously, this allows the pressure of the bone cement dough and of the starting components in the cartridge to be maintained all the way to the three-way valve and, in particular, in the mixer, if any is present. As a result, the time that elapses between the three-way valve being opened and the bone cement dough exiting again is very short. Accordingly, the pressure of the cartridge is maintained between the three-way valve and the dispensing plunger, when the three-way valve is closed, whereas rapid pressure relief of the extended dispensing opening is attained between the three-way valve and the application opening since the bone cement dough flows off through the three-way valve in the closed position. The collecting container is provided to ensure that the bone cement dough does not contaminate the surroundings or the user, and prevents the bone cement dough from dripping out through the three-way valve. Theoretically, it may be sufficient to retain the bone cement dough. The collecting container can just as well be flexible and/or elastic and can expand when it takes up the bone cement dough exiting from the three-way valve.

The storage and mixing device thus contains an emergency relief valve in the form of the three-way valve which may stop the extrusion process instantaneously once the bone cement dough starts to flow into undesired regions of the vertebral body. Said emergency relief valve acts as a pressure relief for the extended dispensing opening of the storage and mixing device, in which the trailing pressure of the bone cement dough from the upstream regions is blocked and simultaneously the bone cement dough situated upstream of the emergency relief valve is relieved of pressure, by opening a channel that leads into the collecting container into which the bone cement dough can exit until the pressure in the hose and/or in the trocar is relieved.

Said storage and mixing devices can preferably be provided appropriately such that the collecting container is impermeable to the bone cement dough towards the outside, preferably in that the collecting container is liquid-tight or liquid-tight and gas-tight. The invention can also provide the collecting container to comprise a volume that is at least as large as half the volume of the extended dispensing opening, in particular of the hose and, if applicable, of the trocar, preferably at least as large as the volume of the extended dispensing opening, in particular of the hose and, if applicable, of the trocar.

The invention can provide a mixer for mixing of the bone cement, in particular a static mixer, between the cartridge and the extended dispensing opening or in the extended dispensing opening or between the cartridge and the three-way valve, whereby the three-way valve is preferred to be arranged between the mixer and the extended dispensing opening. In the latter case, the invention can provide the three-way valve, being in the first position, to provide a fluid connection between the application opening and the mixer and, being in the second position, to close the passage to the mixer.

Referring to preferred storage and mixing devices according to the invention comprising a hose, the hose is flexible, at least regions thereof. The invention can also provide the extended dispensing opening to end in a connector having an internal thread, in particular in a Luer system adapter, or in a trocar.

Preferred storage and mixing devices can also be characterized in that the receptacle comprises a connecting element. In one embodiment the connecting element may be at least one hollow needle or at least one tube arranged on the front side of the receptacle and the receptacle is connected or connectable to the internal space of the cartridge through the connecting element in liquid-conducting manner, and whereby the receptacle is closed in liquid-tight manner except for the connecting element.

As a result, a connection between the receptacle and the cartridge can be attained specifically through the receptacle. Moreover, the connecting element is removed from the cartridge along with the receptacle being removed and can therefore not cause any disturbance later on when the bone cement is extruded from the cartridge.

Moreover, the invention can provide that the monomer liquid container is openable in the inside of the receptacle through a motion of the feed plunger in the direction of the front side of the receptacle, preferably is break-openable or tear-openable.

As a result, the monomer liquid container is openable through the axial linear motion of the feed plunger. Accordingly, an extrusion device with only one axial linear drive can be used in order to open the monomer liquid container and to press the monomer liquid into the cartridge and also to extrude the bone cement dough from the cartridge.

According to a refinement, the storage and mixing device according to the invention can provide the monomer liquid container to be a glass ampoule, a plastic ampoule, a plastic film bag or an aluminum-plastic compound bag that is openable in the inside of the receptacle.

Said monomer liquid containers are well-suited for long-term storage of monomer liquid and are also easy to break open or tear open in the receptacle.

Preferably, the invention can provide that the first securing element and the second securing element are securable to a single holder of an extrusion device, in particular of a cartridge gun.

As a result, a single extrusion device can be used in order to drive both the feed plunger and the dispensing plunger. This simplifies the operation of the storage and mixing device.

Likewise, the invention can preferably provide the internal space of the cartridge to be cylindrical and can provide the external circumference of the dispensing plunger to match the internal space of the cartridge such that the dispensing plunger seals the internal space of the cartridge on its rear side with respect to the outside.

As a result, the design of the storage and mixing device is simplified and is made inexpensive. Moreover, the dispensing plunger comprising said geometry cannot lodge as easily in the internal space of the cartridge such that the storage and mixing device is made less error-prone.

The cartridge is preferred to be tube-shaped.

Moreover, the invention can provide the internal space of the receptacle to be cylindrical and can provide the external circumference of the feed plunger to match the internal space of the receptacle such that the feed plunger seals the internal space of the receptacle on its rear side with respect to the outside.

As a result, the design of the storage and mixing device is simplified and is made inexpensive. Moreover, the feed plunger comprising said geometry cannot lodge as easily in the internal space of the receptacle such that the storage and mixing device is made less error-prone.

The receptacle is preferred to be tube-shaped.

The invention can also provide, on the front side of the cartridge, a dispensing opening through which the content of the cartridge is expellable the dispensing plunger.

In such an exemplary embodiment, a dispensing opening is provided on the side opposite from the dispensing plunger. As a result, the flow resistance of the viscous bone cement dough is kept low.

In order to be able to drive the storage and mixing device independent of any external supply, the invention can provide that the storage and mixing device is operable by a manually operable extrusion device and that the dispensing plunger is movable in the cartridge by manual force such that the bone cement dough is extrudable, and that the feed plunger is movable in the receptacle by manual force such that the monomer liquid container is openable and the monomer liquid is pressable into the internal space of the cartridge.

As a result, the storage and mixing device can be used reliably even in the absence of motors and external energy sources.

Moreover, the invention can provide the cross-section of the internal space of the cartridge to be at most 4 cm², preferably at most 2.5 cm², particularly preferably at most 1.2 cm².

In such exemplary embodiments, it can be made sure that the forces on the inside of the cartridge for extrusion of the viscous bone cement dough do not get excessive. As a result, not only can the design be simplified, but it also becomes feasible to use a manually driven extrusion device.

A preferred refinement can provide the cartridge to be closed off on the front side by a cartridge head, whereby an outlet opening is situated in the cartridge head and the outlet opening is closed by a closure which is impermeable to the cement powder in the cartridge and which is permeable to gas, in particular a pore filter, whereby the closure is preferably openable through an axial pressure load or by action of a manual force.

As a result, the inside of the cartridge and the cement powder can be sterilized by a sterilizing gas, such as ethylene oxide. In addition, the cement powder cannot get outside and be lost, which, if it were otherwise, would falsify the mixing ratio of cement powder and monomer liquid and therefore the composition of the bone cement dough.

For long-term storage of the cement powder, the invention can provide the dispensing plunger to be impermeable to the cement powder particles and comprise a feedthrough which is permeable to the monomer liquid, whereby the feedthrough is preferably arranged on the side of the dispensing plunger facing the rear side, and the feedthrough is closable.

This prevents the cement powder from advancing in the direction of the receptacle and thereby impeding the transfer of monomer liquid.

The invention can further provide the receptacle to be closed or closable on the rear side by the feed plunger in liquid-tight manner, preferably to be closed or closable in liquid-tight and gas-tight manner.

This prevents the monomer liquid from leaking from the receptacle towards the back. This allows the desired mixing ratio to be assured. Moreover, contamination of the surroundings by the monomer liquid can be prevented.

According to a preferred embodiment, the present invention can provide the cartridge to be hollow cylinder-shaped and the second securing element to be provided to be connected to an extrusion device, whereby a cartridge head closes the front side of the hollow cylinder-shaped cartridge, whereby an outlet opening is arranged in the cartridge head, and whereby the outlet opening connects the outside of the cartridge head to the inside of the cartridge head in gas-permeable manner, whereby a dispensing tube is connected to the outlet opening on the cartridge head, whereby a puncturable membrane is provided that closes the rear side of the dispensing plunger in liquid-impermeable manner, and the receptacle is hollow cylinder-shaped, whereby a porous disk, which is permeable to gases and liquids, is provided in a connecting plunger in the receptacle, and at least one hollow needle is arranged on the front side of a connecting plunger, the at least one hollow needle being connected to the rear side of the porous disk in liquid-permeable manner by a channel.

This design simplifies the storage and mixing device and makes it particularly inexpensive.

The invention can provide for the monomer liquid container to be opened by exposure to pressure. For example, the monomer liquid container may be openable by applying a linearly acting force, such as the pressure exerted by the extrusion device.

Preferred storage and mixing devices can be characterized in that the internal diameter of the cartridge is smaller than or equal to the internal diameter of the receptacle, preferably the internal diameter of the cartridge is smaller than the internal diameter of the receptacle.

In such a non-limiting embodiment, the force required to extrude the viscous bone cement dough from the cartridge can be reduced by the longer stroke without an unnecessarily large stroke being required for opening the monomer liquid container and for extruding the monomer liquid from the receptacle.

According to a refinement, the invention can provide the volume of the monomer liquid in the monomer liquid container to be at least as large as the volume of the air-filled intervening spaces between the cement powder particles in the cartridge, preferably to be at least as large as the volume of the liquid conduits between the internal space of the cartridge and the internal space of the receptacle plus the volume of the air-filled intervening spaces between the cement powder particles in the cartridge.

As a result, it can be made sure that all of the cement powder can be wetted by the monomer liquid and that, thus, a homogeneous bone cement dough is produced.

In this context, the invention can preferably provide intervening spaces between the cement powder particles in the cartridge to account for between 22% by volume and 40% by volume of the bulk powder of the cement powder.

To make it easier to meet the hygienic requirements, the invention can provide for at least one ventilation opening to be arranged in the wall of the receptacle, whereby the ventilation opening connects the internal space of the receptacle, in which the monomer liquid container is arranged, to the surroundings, whereby the at least one ventilation opening is preferably arranged sufficiently closely in the area of the feed plunger such that it is closed by a motion of the feed plunger in the direction of the front side of the receptacle before the monomer liquid container is opened through the motion of the feed plunger.

As a result, the internal space of the receptacle can be sterilized with a sterilizing gas. Concurrently, the monomer liquid cannot exit from the internal space of the receptacle, when the at least one ventilation opening is closed by the feed plunger moving in the direction of the front side of the receptacle and before the monomer liquid container is opened by the motion of the feed plunger, for example is squashed, splintered or torn open by the feed plunger in the internal space of the receptacle.

Preferably the invention can also provide the cartridge to have a compressive strength of more than 10 bar, preferably of more than 50 bar, particularly preferably of more than 70 bar.

What this attains is that the large forces occurring while extruding the viscous bone cement dough from the cartridge do not deform the walls of the cartridge to the extent that it would become leaky or block the motion of the dispensing plunger.

The objects underlying the present invention are also met by a method for the production of a bone cement dough, in particular of a pasty polymethylmethacrylate bone cement dough, whereby the bone cement dough is produced from a cement powder and a monomer liquid using a storage and mixing device, whereby the storage and mixing device comprises a receptacle containing a monomer liquid container with the monomer liquid in it and a cartridge containing the cement powder, whereby the receptacle is secured or is securable to the rear side of the cartridge, characterized by the following steps taking place in the order given:
a) inserting the storage and mixing device in an extrusion device, whereby the extrusion device comprises a pestle that is propellable in axial direction;
b) propelling a feed plunger, supported such as to be mobile in the receptacle on the rear side thereof, in the direction of the cartridge by the pestle, whereby the motion of the feed plunger opens the monomer liquid container and presses the monomer liquid from the monomer liquid container into the cartridge, whereby the cement powder mixes with the monomer liquid in the internal space of the cartridge;
c) separating the receptacle from the cartridge, whereby the cartridge remains in the extrusion device or is reinserted into the extrusion device after the separation;
d) propelling a dispensing plunger, supported in the cartridge on the rear side thereof such as to be mobile, in the direction of the front side of the cartridge by the pestle, whereby the motion of the dispensing plunger expels the mixture consisting of the cement powder and the monomer liquid from the cartridge in the form of the bone cement dough.

In this context, the invention can provide the method to be implemented with a storage and mixing device according to the invention.

Moreover, the invention can provide a closable feedthrough on the rear side of the cartridge through which the monomer liquid is pressed into the inside of the cartridge in step b), whereby the closable feedthrough is being closed when the receptacle is separated from the cartridge in step c) or closes self-actingly during the separation.

The effect of designing the feedthrough to be closable is that, after feeding the monomer liquid into the cartridge, no monomer liquid can exit through same on the rear side of the cartridge after the receptacle is detached from the cartridge. In such an embodiment, contamination and/or soiling of the surgical area with the monomer liquid is prevented.

Preferably, the invention can provide a first securing element on the rear side of the receptacle and a second securing element on the rear side of the cartridge, whereby, during the insertion of the storage and mixing device in step a), the storage and mixing device is secured to a holder of the extrusion device by the first securing element, and the cartridge is secured to the holder of the extrusion device by the second securing element in step c).

In such an embodiment, both the receptacle and the cartridge are securable to the same holder of an extrusion device, such as a cartridge gun, and therefore the receptacle and the cartridge can be extruded with the same extrusion device. The term cartridge gun shall be understood to also include caulking guns. It is preferred to use a manually-driven extrusion device or cartridge gun. The invention can therefore preferably provide the first securing element and the second securing element to be suitable and provided to be secured to a holder of an extrusion device.

In order to simplify the implementation of the method, the invention can provide the receptacle to be separated from the cartridge in step c) by a screw connection, a plug connection, a snap-in connection or a bayonet lock being undone.

As a result, a stable connection of the receptacle and the cartridge is attained when both are connected and/or secured to each other.

Moreover, the invention can provide a connecting plunger to be arranged on the front side of the receptacle, whereby the monomer liquid container is arranged between the connecting plunger and the feed plunger, whereby the connecting plunger is driven in the direction of the cartridge in step b), while the feed plunger is propelled and before the monomer liquid is pressed into the cartridge, whereby a passage into the cartridge is thus opened such that the internal space of the cartridge and the internal space of the receptacle are connected to each other between the feed plunger and the connecting plunger in liquid-permeable manner, whereby, preferably in thereby or subsequently, the monomer liquid container is opened through a motion of the feed plunger against the connecting plunger, in particular a plastic or glass ampoule as the monomer liquid container is crushed between the feed plunger and the connecting plunger such that the monomer liquid is available in the internal space of the receptacle to be pressed into the cartridge.

What this attains is that the fluid connection between the internal space of the cartridge and the internal space of the receptacle is opened only upon the connecting plunger being moved. When the plunger is moved, the internal space of the cartridge can be prevented from being opened inadvertently. Moreover, the motion of the connecting plunger can open the closable passage such that the closure of the closable passage is openable by the same linear drive that is also used to drive the feed plunger and the dispensing plunger.

Preferably, the internal space of the cartridge and the internal space of the receptacle between the feed plunger and the connecting plunger are thus connected to each other in a liquid-permeable manner and cement powder-impermeable manner. It is also preferred for the monomer liquid container to be compressed or crushed into small fragments between the connecting plunger and the feed plunger.

According to a refinement of the method according to the invention, the invention can provide the mixture of the cement powder and monomer liquid in step d) to be pressed out of the cartridge and through an extended dispensing opening, a dispensing tube, a hose, a trocar and/or a static mixer, whereby the bone cement dough is produced therein or earlier in the cartridge.

The method can be used particularly well for vertebroplasty. Due to the linear two-part design and the ability to design the cartridge to be very narrow, it is feasible to utilize a manually-driven method with a manually driven extrusion device although the bone cement dough is very viscous and although the dispensing opening is long, possibly including trocar and hose, which increase the flow resistance even further. This is the case because the stroke distance with which the storage and mixing device is driven is very long and, due to the elongated design and the ensuing relatively small internal diameter of the internal space of the cartridge, a long stroke will extrude only a small amount of the bone cement dough through the extended dispensing opening.

Moreover, the invention can provide a three-way valve, which is operable from outside, to be arranged in the extended dispensing opening or downstream from the cartridge, whereby the three-way valve is brought into a first position or is in the first position, whereby the three-way valve, being in the first position, provides a continuous connection from the internal space of the cartridge through the extended dispensing opening, and an extrusion of the cartridge by the extrusion device takes place in step d), whereby the bone cement dough mixed from the cement powder and the monomer liquid is pressed through the three-way valve and through the extended dispensing opening, and a subsequent step e) takes place, in which the three-way valve is transitioned into a second position, whereby the three-way valve, being in the second position, stops the flow from the cartridge through the three-way valve and a part of the pressurized bone cement dough downstream from the three-way valve in the extended dispensing opening is pressed through the three-way valve into a collecting container.

Moreover, the invention can provide the three-way valve to be transitioned to the first position again in a step f) after step e) and, the bone cement dough to be guided again through the three-way valve through the extended application opening, whereby it is preferred for steps d), e), and f) to be repeated once or multiple times in the order given.

The invention is based on the surprising finding that subdividing the storage and mixing device into two parts and having the two identical or congeneric securing elements for connecting the extrusion device allows a conventional extrusion device to be used, whereby the first stroke of the extrusion device initially opens the monomer liquid container and presses the monomer liquid through a connection of the two parts, namely of the receptacle and the cartridge, into the cement powder, and subsequently one part, namely the receptacle, is separated from the other part of the storage and mixing device, and the extrusion device subsequently dispenses bone cement dough from the cartridge by the second stroke. As a result, a particularly thin cartridge with a small internal diameter can be used such that the highly viscous bone cement dough can still be extruded and applied against the resistance of a hose without the extrusion device having to be designed to be very elongated with a particularly long pestle. As a result, the storage and mixing device and the method can also be used for vertebroplasty.

Both the method according to the invention of the storage and mixing device according to the invention are based on a common idea in that an initially connected two-part storage and mixing device is used to open the monomer liquid container and to transfer the monomer liquid into the cartridge with an extrusion device, followed by separating the cartridge from the remaining storage and mixing device, and in that the bone cement dough can be expelled from the cartridge with the same extrusion device. For this purpose, it is preferred to use identical securing elements on the cartridge and on the receptacle, that are being connected to the same opposite securing elements on the extrusion device. With an appropriate design of the storage and mixing device and/or suitable process steps, a manually driven extrusion device can be used. Moreover, what suitable measures according to the invention attain is that no monomer liquid and no bone cement dough exit from the storage and mixing device and contaminate the environment during the storage and mixing of the bone cement dough. Lastly, the measures according to the invention allow the storage and mixing device to be designed as a hygienic disposable product for single use.

The particular advantage of the storage and mixing device according to the invention is that conventional manually-driven extrusion devices that are common for normal PMMA cements can be used to extrude a two-component spine cement and/or the bone cement dough for vertebroplasty through a thin hose into the trocar. The augmentation of vertebral bodies takes place under permanent x-ray control. Having a hose between the trocar and the applicator allows the physician to not have to work with his hands within the range of the x-rays. No complex expensive hydraulic application devices are required in this context.

Moreover, it has been evident, surprisingly, that the connection between the two parts being closable on the rear side of the cartridge allows the cartridge, after supply of the monomer liquid into the cement powder, to be separated from the remaining storage and mixing device without remaining monomer liquid exiting on the rear side of the cartridge. Simultaneously, the system is rapidly ready for use and is operable without any greater effort. The storage and mixing device can be used as a hygienic disposable product since it can be manufactured largely from plastics and since all parts including the internal spaces and the cement powder can be sterilized with ethylene oxide.

It is to be understood that both the foregoing general description and the following detailed description are exemplary, but are not restrictive, of the invention.

### BRIEF DESCRIPTION OF THE DRAWING

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a schematic cross-sectional view of one embodiment of a storage and mixing device;

FIG. 2 is a schematic side view of the storage and mixing device shown in FIG. 1.

FIG. 3 is four schematic cross-sectional views of the storage and mixing device shown in FIGS. 1 and 2 on top of each other for illustration of the workflow of one embodiment of the method;

FIG. 4 is a schematic cross-sectional view through the front part of the storage and mixing device shown in FIGS. 1 to 3 with an extended dispensing opening connected to it;

FIG. 5 is three schematic perspective views of parts of the storage and mixing devices shown in FIGS. 1 to 4;

FIG. 6 is a schematic cross-sectional view of a three-way valve for an extended dispensing opening shown in FIG. 4;

FIG. 7 is a schematic cross-sectional view of the three-way valve shown in FIG. 6 with a sectional plane perpendicular to the flow direction of the bone cement dough;

FIG. 8 is a schematic cross-sectional view of the connection of the two parts of the storage and mixing device during its use according to the 2nd figure from the top in FIG. 3;

FIG. 9 is a schematic cross-sectional view of the connection of the two parts of the storage and mixing device before dispensation according to the 4th figure from the top in FIG. 3;

FIG. 10 is a schematic perspective partial view (top) and a schematic partial cross-sectional view (bottom) of the rear side of the rear part of one embodiment of a storage and mixing device in the starting state;

FIG. 11 is two schematic perspective cross-sectional views through a valve system for an alternative storage and mixing device according to the invention, namely a three-way valve in closed position (FIG. 11 top) and in open position (FIG. 11 bottom);

FIG. 12 is two schematic cross-sectional top views through the valve system according to FIG. 11, namely the three-way valve in open position (FIG. 12 bottom) and in closed position (FIG. 12 top);

FIG. 13 is a schematic cross-sectional view through a third inventive exemplary embodiment of a storage and mixing device; and

FIG. 14 is a magnified detail view of the connection of the two parts of the third exemplary embodiment according to FIG. 13 before the start of the extrusion process.

### DETAILED DESCRIPTION

The features and benefits of the disclosed material transport apparatus are illustrated and described by reference to exemplary embodiments. The disclosure also includes the drawing, in which like reference numbers refer to like elements throughout the various figures that comprise the drawing. This description of exemplary embodiments is intended to be read in connection with the accompanying drawing, which is to be considered part of the entire written description. Accordingly, the disclosure expressly should not be limited to such exemplary embodiments illustrating some possible non-limiting combinations of features that may exist alone or in other combinations of features.

In the description of embodiments, any reference to direction or orientation is merely intended for convenience of description and is not intended in any way to limit the scope of the present invention. Relative terms such as “front”, “rear”, “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be construed or operated in a particular orientation. Terms such as “attached,” “affixed,” “connected,” “coupled,” “interconnected,” and similar terms refer to a relationship wherein structures are secured or attached to one another either directly or indirectly through intervening structures, as well as both moveable or rigid attachments or relationships, unless expressly described otherwise.

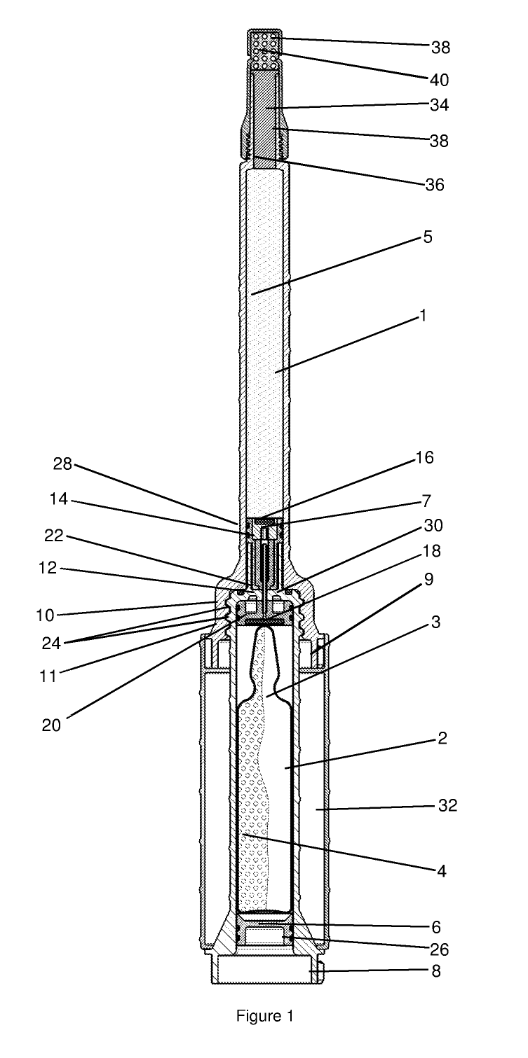

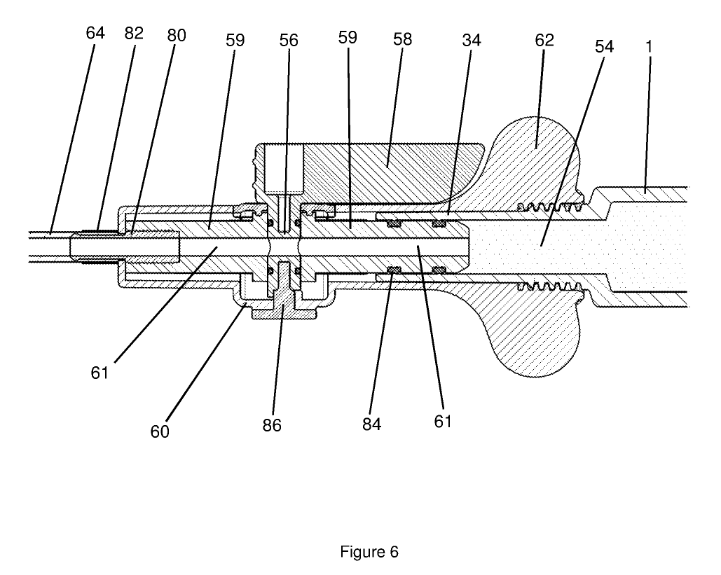

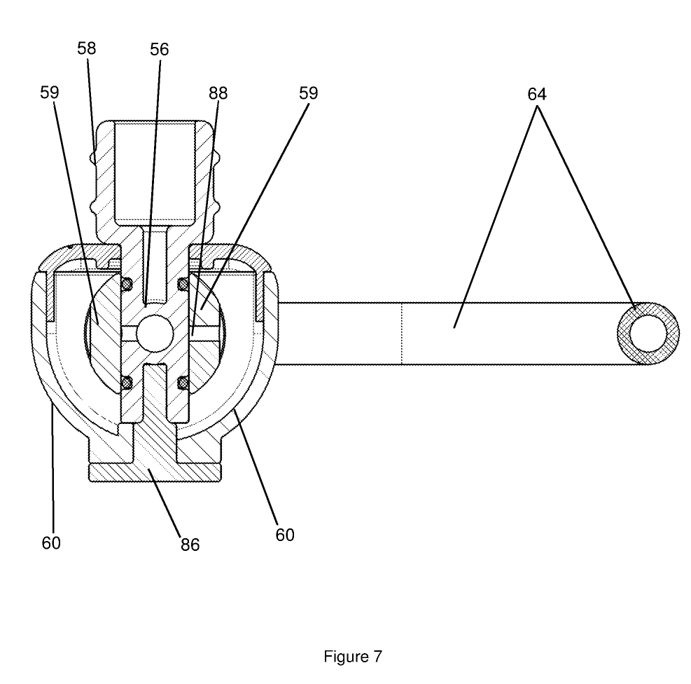

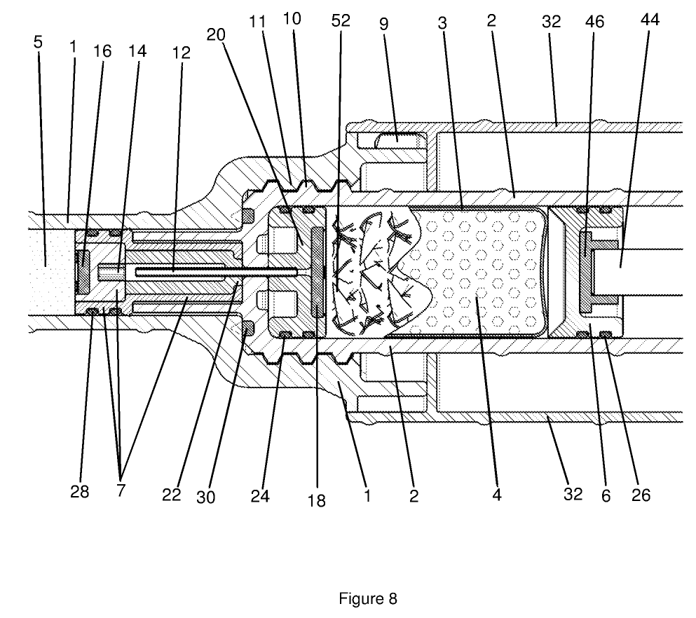

FIGS. 1 to 5 show various schematic views of a first exemplary storage and mixing device according to the invention. FIGS. 6 and 7 show schematic cross-sectional views as detail views through a valve system that can be used as part of the inventive storage and mixing device according to FIGS. 1 to 5. FIGS. 8 to 10 show further detail views of the inventive storage and mixing device according to FIGS. 1 to 5.

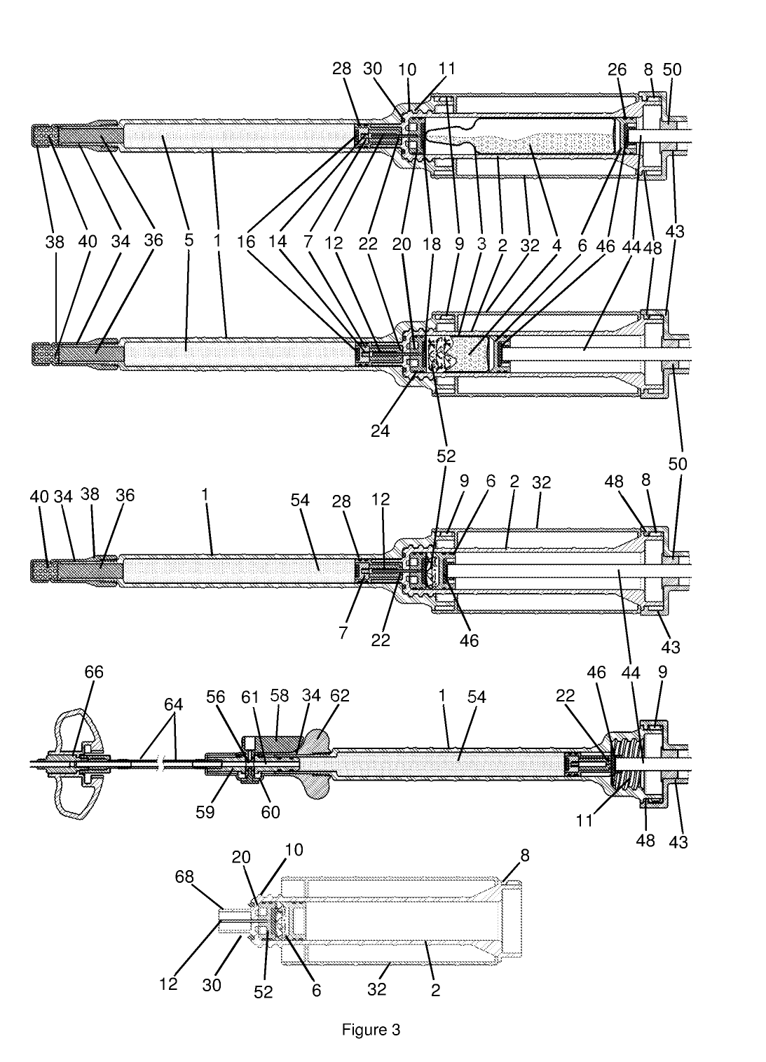

The storage and mixing device has, as front part (on the top in FIGS. 1, 2, and 4, on the left in FIGS. 3, 5, 6, 8, and 9), a cartridge 1 made of plastics with a cylindrical internal space. The cartridge 1 is detachably connected to a receptacle 2 made of plastics for a glass ampoule 3 (or plastic ampoule 3). The receptacle 2 also comprises a cylindrical internal space into which the glass ampoule 3 is plugged. The monomer liquid 4 is situated in the glass ampoule 3. A cement powder 5 is filled or, preferably, pressed into the internal space of the cartridge 1. The monomer liquid 4 and the cement powder 5 are the starting components 4, 5 of a PMMA bone cement that can be produced with the storage and mixing device. Due to the glass ampoule 3, the monomer liquid 4 can be stored in the receptacle 2 and therefore in the storage and mixing device for very long times.

A feed plunger 6 made of plastics that is mobile in longitudinal direction in the cylindrical internal space of the receptacle 2 is arranged in the receptacle 2. The feed plunger 6 is arranged in the area of the rear side of the receptacle 2. The glass ampoule 3 can be compressed and thereby cracked in the receptacle 2 by the feed plunger 6 by pushing the feed plunger 6 in the direction of the front side, i.e. in the direction of the cartridge 1. The feed plunger 6 comprises, on its front side, wipers which may assist with wiping off fragments of the glass ampoule 3 from the internal wall of the receptacle 2.

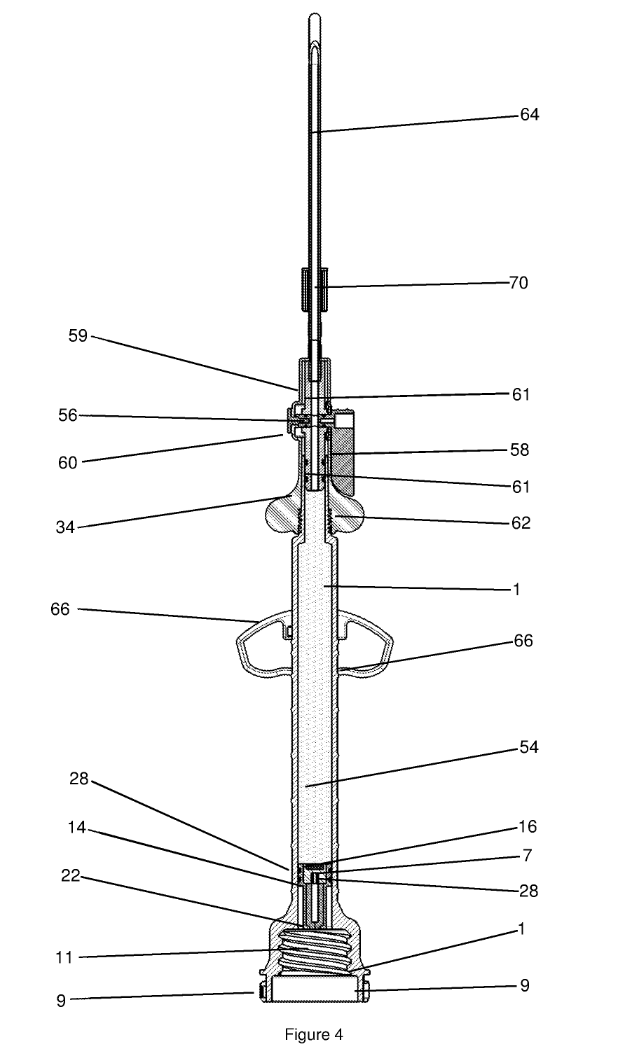

A dispensing plunger 7 made of plastics is arranged in the rear side (toward the bottom in FIGS. 1, 2, and 4, toward the right in FIGS. 3, 5, 6, 8, and 9) of the internal space of the cartridge 1. A securing element 8 is provided on the rear side of the receptacle 2 and can be used to connect the receptacle 2 to an extrusion device 43 (not shown in FIG. 1, but in FIG. 3). A securing element 9 of identical design is provided on the rear side of the cartridge 1. Both the receptacle 2 and the cartridge 1 can therefore be secured to the same extrusion device 43. The securing elements 8, 9 are preferably suitable and provided for the formation of a bayonet lock 8, 9. As a result, the feed plunger 6, which is freely accessible from the rear side of the receptacle 2, can be driven forward in the direction of the front side using the same extrusion device 43 as the dispensing plunger 7, which is freely accessible from the rear side of the cartridge 1, when the cartridge 1 is not connected to the receptacle 2 (see, for example, FIG. 4).

The cartridge 1 and the receptacle 2 are detachably connected or are detachably connectable to each other via an external thread 10 on the front side of the receptacle 2 and an internal thread 11 on the rear side of the cartridge 1. The receptacle 2 and the cartridge 1 are connected to each other in liquid-permeable manner with respect to the monomer liquid 4 by a tube 12 made of a metal or of plastics or a feedthrough 14 in the dispensing plunger 7. The feedthrough 14 through the dispensing plunger 7 merges through a pore filter 16, which is impermeable to the cement powder 5, but permeable to the monomer liquid 4, into the internal space of the cartridge 1. Before entering the internal space of the cartridge 1, the feedthrough 14 branches into multiple arms, whereby the arms are not shown in the section according to FIG. 1 since they are not situated in the central plane of the storage and mixing device that is shown and shown in section therein.

A filter 18, by which the fragments of the glass ampoule 3 can be retained, is arranged in the connection to the tube 12 in the receptacle 2. Instead of the filter 18 or in addition to the filter 18, a screen can be provided just as well or also. The filter 18 is arranged in a connecting plunger 20 to which the tube 12 is secured as well. In this embodiment, the connecting plunger 20 touches against the front side of the receptacle 2 on the inside and serves only the purpose to facilitate the assembly of the storage and mixing device. Theoretically, the tube 12 and the filter 18 can just as well be affixed to the receptacle 2 and the connecting plunger 20 can be omitted.

The tube 12 is guided through a valve with three sealing lips 22 into the feedthrough 14 through the dispensing plunger 7. The three sealing lips 22 are implemented by a Y-shaped incision in an elastic plastic plug, whereby the plastic stopper forms the rear-side part of the dispensing plunger 7. In the starting state, the tube 12 is guided through said sealing lips 22, and the passage 14 is sealed in liquid-tight manner in the direction of the rear side by the sealing lips 22 touching against the tube 12. When the tube 12 is pulled out of the plug and/or the sealing lips 22, the sealing lips 22 seal the dispensing plunger 7, and thus the cartridge 1, on the rear side in liquid-tight manner.

The cylindrical connecting plunger 20 has an external circumference that matches the cylindrical geometry of the internal space of the receptacle 2 and is sealed by two circumferential seals 24 in liquid-tight manner with respect to the internal wall of the receptacle 2. Likewise, the feed plunger 6 is sealed in liquid-tight manner with respect to the internal wall of the receptacle 2 by two circumferential seals 26 and the dispensing plunger 7 is sealed in liquid-tight manner with respect to the internal wall of the cartridge 1 by two circumferential seals 28. Moreover, the cartridge 1 and the receptacle 2 are sealed with respect to each other by a seal 30 that is secured to the front surface of the receptacle 2, when the cartridge 1 and the receptacle 2 are secured to each other. The purpose of all of said seals 24, 26, 28, 30 is to prevent monomer liquid 4 or bone cement from exiting in order to prevent contamination of the surroundings (the OR theatre and the user). For this purpose, the seals 24, 26, 28, 30 can consist of rubber.

In addition, the receptacle 2 is surrounded by a housing 32 made of plastics, whereby the housing 32 is connected to the receptacle 2 and covers, on the front side, the securing element 9 of the cartridge 1, when the cartridge 1 is connected to the receptacle 2. The cartridge 1, the receptacle 2, and the housing 32 can be produced through injection molding.

The front side of the cartridge 1 merges into a dispensing tube 34 that comprises an external thread. A pore filter 36 that is impermeable to the cement powder 5, but is permeable to gases is arranged in the inside of the dispensing tube 34. A cap 38 is secured to the external thread of the dispensing tube 34, whereby the front part of the cap 38 is filled with a Styrofoam or foam 40. Two wings 42 are provided on the cap 38 such that the cap 38 can be unscrewed conveniently from the dispensing tube 34 in the way of a wing screw. The cap 38 comprises lateral openings. Due to this design, the inside of the cartridge 1 and the cement powder 5 can be sterilized with the aid of ethylene oxide, since the openings in the cap 38, the Styrofoam or foam 40, the pore filter 36, and the intervening spaces between the powder particles of the cement powder 5 are permeable to air. Concurrently, air can be pressed out of the receptacle through the cement powder 5, the pore filter 36, the Styrofoam or foam 40, and the openings in the cap 38, when the feed plunger 6 is pressed in the direction of the receptacle 2.

The cement powder 5 is enclosed in the cartridge 1, since all openings are closed by the pore filters 16, 36 such as to be impermeable to the cement powder 5. The content of the cartridge 1 can be sterilized by evacuation and rinsing with ethylene oxide in this context. As a result, the storage and mixing device is also well-suited for long-term storage of the cement powder 5.

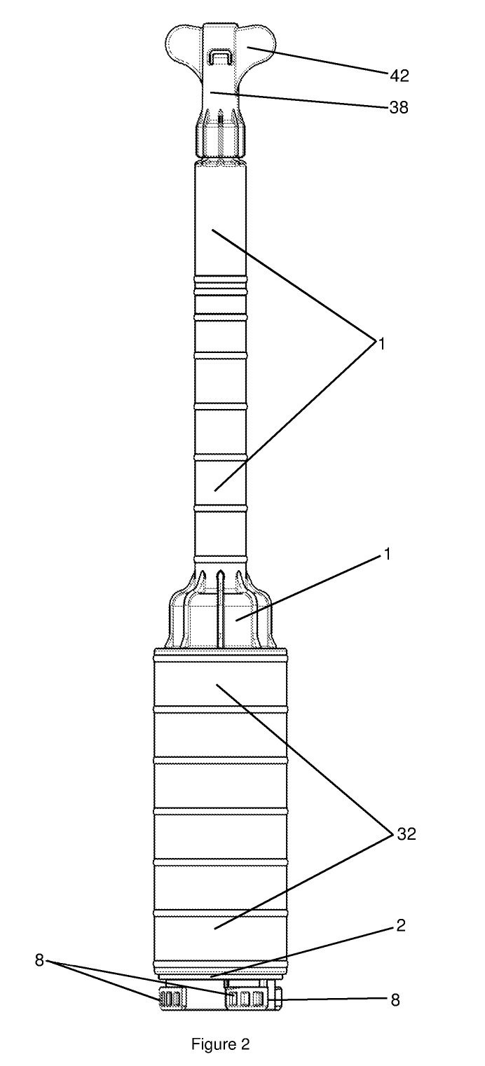

FIG. 3 shows four schematic cross-sectional views of the storage and mixing device according to FIGS. 1 and 2 on top of each other for illustration of the workflow of a method according to the invention. At the outset of the method, the storage and mixing device is in the starting state that is also shown in FIG. 1. Accordingly, the cartridge 1 and the receptacle 2 of the storage and mixing device are initially connected to each other through the threads 10, 11. Being in this state, the storage and mixing device is inserted into an extrusion device 43 in the form of a conventional cartridge gun and is secured to the extrusion device 43 using the securing element 8 (see topmost depiction in FIG. 3). Only the front part of the extrusion device 43 is shown. The extrusion device 43 comprises a pestle 44 that can be propelled linearly. Preferably, the pestle 44 is driven manually. The pestle 44 ends, on its front side, in a cup 46 that can be used to push onto the feed plunger 6. For this purpose, the extrusion device 43 is connected to the rear side of the receptacle 2 through an opposite securing element 48 such that the cup 46 pushes onto the feed plunger 6 and propels it in the direction of the cartridge 1, when the pestle 44 is propelled forward. For this purpose, the pestle 44 is supported such as to be linearly mobile with respect to a bearing 50 and, by means of it, with respect to the opposite securing element 48 and therefore with respect to the receptacle 2.

Operating the extrusion device 43 propels the pestle 44 and, the pestle 44 moves the feed plunger 6 in the direction of the cartridge 1. Since the plastic or glass ampoule 3 touches against the connecting plunger 20 on its front side, the internal space of the receptacle 2 decreases in size and the glass ampoule 3 fractures and the monomer liquid 4 exits from the glass ampoule 3 into the internal space of the receptacle 2. The situation is shown in FIG. 3, 2nd figure from the top and in a magnified detail view in FIG. 8. Supernatant air from the receptacle 2 is pushed through the filter 18, the feedthrough 14, the pore filter 16, through the intervening spaces between the particles of the cement powder 5, through the pore filter 36, through the foam 40, and out of the openings in the cap 38 out of the storage and mixing device.

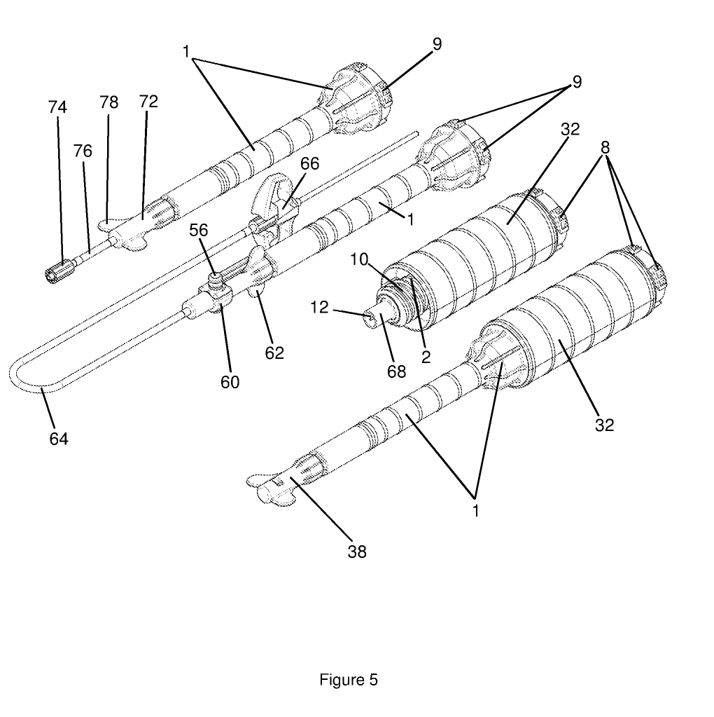

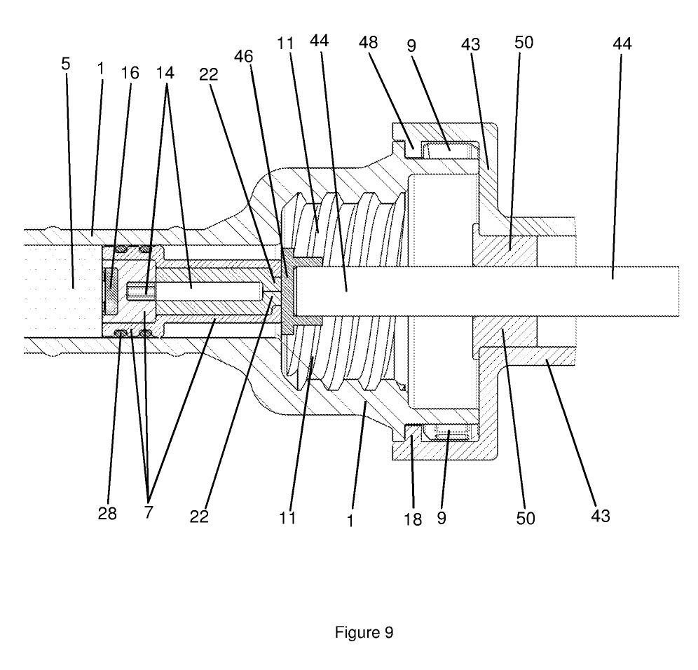

Lastly, only small fragments 52 of the glass ampoule 3 remain and are retained by the filter 18 and remain in the receptacle 2. The monomer liquid 4 is pressed through the filter 18, the feedthrough 14, and the pore filter 16 into the cement powder 5 and there starts to react with the cement powder 5 such that the bone cement dough 54 is produced from the mixture 54. This situation is shown in FIG. 3, third figure from the top. In addition, the cartridge 1 with the extended dispensing opening is also shown in FIG. 4 and FIG. 5. At this stage, the storage and mixing device is taken out of the extrusion device 43. The receptacle 2 and the housing 32 are unscrewed from the cartridge 1. In addition, the cap 38 with the pore filter 36 and the foam 40 is unscrewed and an extended dispensing opening is screwed onto the dispensing tube 34. Subsequently, the cartridge 1 with the extended dispensing opening is secured again to the extrusion device 43. Earlier, the extrusion device 43 was reset, i.e. the pestle 44 was pushed back again and transitioned into the starting position. After the tube 12 is pulled out, the sealing lips 22 close by their own action such that no monomer liquid 4 still be present in the feedthrough 14 can exit on the rear side of the cartridge 1, when the cartridge 1 is separate from the receptacle 2 (also see the detail view of the detail magnification according to FIG. 9).

The opposite securing element 48 of the extrusion device 43 engage the securing element 9 on the rear side of the cartridge 1 in order to secure the extrusion device 43 to the cartridge 1. Since the securing element 9 on the cartridge 1 and the securing element 8 on the receptacle 2 are identical, both can be secured to the same opposite securing element 48 of the same extrusion device 43. This simplifies the design and enables the use of conventional extrusion devices 43. In this context, the securing element 8, 9 and the opposite securing element 48 form a bayonet lock.

The extended dispensing opening comprises a three-way valve 56 that can be operated manually from outside through a T-handle 58. The three-way valve 56 is seated in a tight fit in a tube 59 that forms a valve seat 59. FIGS. 6 and 7 show schematic cross-sectional views as detail views through a valve system that can be used as part of the inventive storage and mixing device according to FIGS. 1 to 5. A collecting container 60, which is closed with respect to the outside and serves for receiving bone cement dough 54, is arranged in the area of the three-way valve 56. A passage 61 forming the inside of the tube 59 is provided in the tube 59. The passage 61 in the front side of the tube 59 can be connected to the receptacle 60 via the three-way valve 56 such that pressurized bone cement dough 54 can evade from the front side of the extended dispensing opening to this location such that no more than a small amount of the bone cement dough 54 continues to flow from the extended dispensing opening, when the three-way valve 56 is in said closed position. As a result, the amount of bone cement dough 54 that continues to flow is reduced through the use of the three-way valve 56. The three-way valve 56 is screwed onto the dispensing tube 34 with the aid of wings 62. The front part of the tube 59 is connected to a hose 64 that merges into a trocar 66.

The unscrewed rear-side part of the storage and mixing device, namely the receptacle 2 with the fragments 52 in it and the housing 32, stays behind and can be disposed. The front side of the receptacle 2 has a protective tube 68 provided on it, in which the tube 12 is arranged such that the user cannot get injured as easily on the tube 12.

The bone cement dough 54 can be dispensed through the hose 64 and the trocar 66. For this purpose, the dispensing plunger 7 is propelled in the direction of the dispensing tube 34 by the pestle 44 and the cup 46. When the three-way valve 56 is open, as is shown in FIG. 3 (bottom drawing) and in FIGS. 6 and 7, the bone cement dough 54 is expelled through the dispensing tube 34, through the three-way valve 56 and the passage 61, through the hose 64 and the trocar 66 and can there be applied to the vertebrae of a patient or theoretically be used for further processing. The three-way valve 56 can be operated in order to interrupt the flow of the bone cement dough 54. Pressurized bone cement dough 54 from the hose 64 and the trocar 66 can flow through the three-way valve 56 into the collecting container 60 without contaminating the surroundings. As a result, the flow of the bone cement dough 54 is interrupted rapidly.

The trocar 66 is connected to the hose 64 through a Luer system adapter 70 (see FIG. 4 or second embodiment from the top on the left in FIG. 5). The trocar 66 can just as well be directly connected to the hose 64. The first embodiment on the top left in FIG. 5 shows a variant, in which a connector 72 connects a Luer system adapter 74 via a short hose 76 to the cartridge 1. The connector 72 can be screwed onto the dispensing tube 34 of the cartridge 1 with the aid of wings 78 in the way of a wing screw. For this purpose, the connector 72 comprises a matching internal thread.

The detail view of the detail magnification according to FIG. 6 of the valve system additionally shows that the tube 59 is connected to the hose 64 by an insert 80, which comprises a channel that aligns with the passage 61. For this purpose, the insert 80 is screwed into the tube 59. In order to ensure a pressure-tight connection, the hose 64 is crimped onto the insert 80 by a metal sleeve 82, and the tube 59 is sealed with respect to the internal wall of the dispensing tube 34 by two circumferential seals 84. The underside of the three-way valve 56 is secured with a stopper 86 such that the three-way valve 56 cannot be pulled easily from the valve seat 59 or tube 59. A draining channel 88 can be seen in the valve seat 59 in the sectional plane according to FIG. 7, which is perpendicular to the sectional plane according to FIG. 6 and is situated perpendicular to the passage 61, whereby, with the three-way valve 56 being in the closed position, the bone cement dough 54 can flow from the front side of the extended dispensing opening through said draining channel into the collecting container 60.

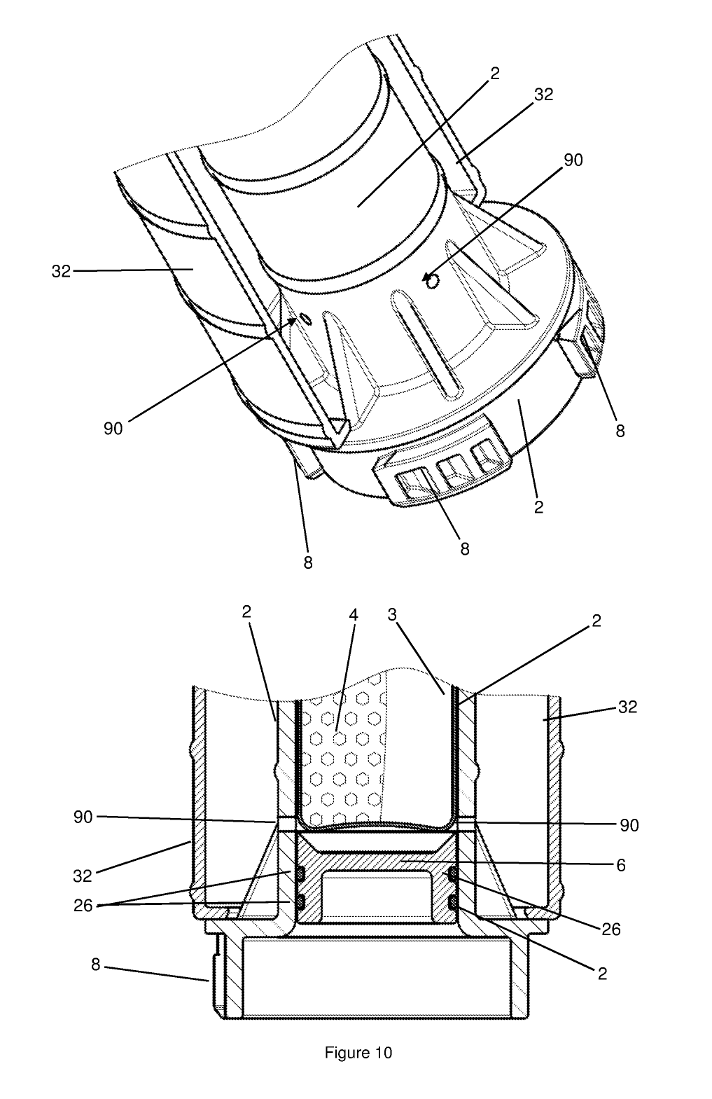

FIG. 10 shows a schematic perspective partial view (top) and a schematic partial cross-sectional view (bottom) as detail view of the rear side of the rear part of a storage and mixing device in the starting state. It can be seen that the wall of the receptacle 2 is provided with multiple ventilation openings 90 through which the internal space of the receptacle 2 can be sterilized with the aid of a sterilizing gas such as ethylene oxide. The ventilation openings 90 are arranged immediately adjacent to the feed plunger 6 such that the feed plunger 6 immediately closes the ventilation openings 90 when it is being propelled in the direction of the cartridge 1. This prevents monomer liquid 4 from exiting through the ventilation openings 90, when the glass ampoule 3 in the receptacle 2 was opened.

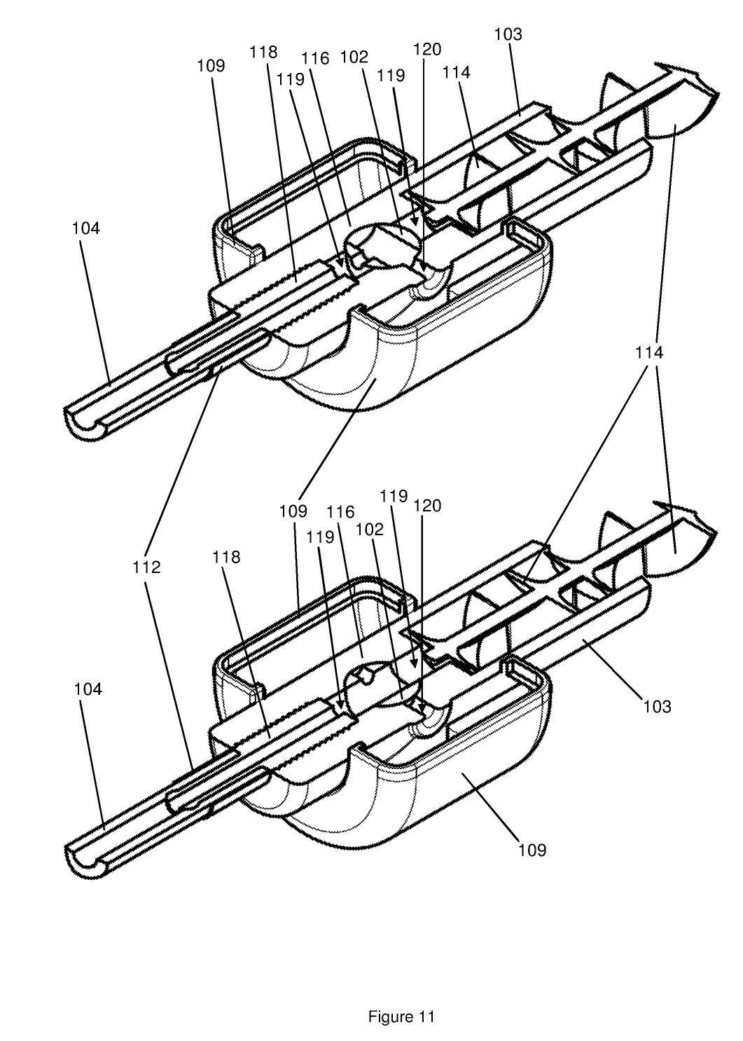

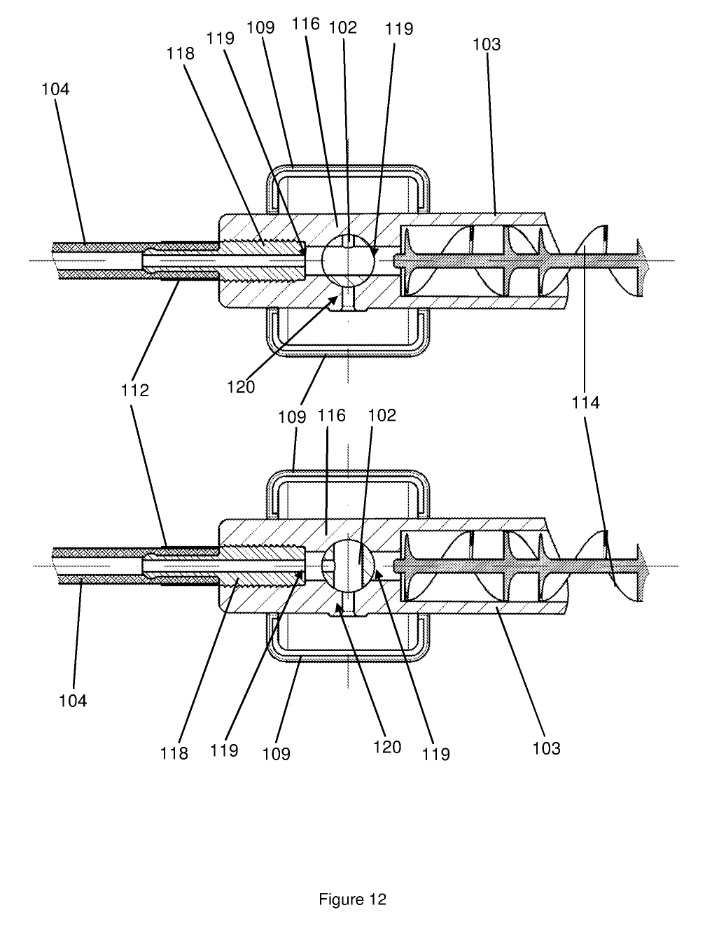

FIGS. 11 and 12 each show two schematic perspective cross-sectional views through an exemplary three-way valve 102 for a second alternative storage and mixing device according to the invention, namely the three-way valve 102 in closed position (FIG. 11 top and FIG. 12 bottom) and in open position (FIG. 11 bottom and FIG. 12 top) for illustration of the mode of function of the three-way valve 102 by the internal design.

The design of the alternative second storage and mixing device according to the invention is the same as that of the preceding first exemplary embodiment according to FIGS. 1 to 10 unless described otherwise or visualized in FIGS. 11 and 12.

A tube 103 is arranged as extended dispensing opening on the front side of a cartridge (not shown in FIGS. 11 and 12, but provided as in the preceding exemplary embodiment). A hose 104 through which the bone cement dough (not shown in FIGS. 11 and 12) can be dispensed is secured to the valve system downstream of the valve system. To make sure that the bone cement dough cannot exit in uncontrolled manner, a collection container 109 is provided analogous to the embodiment according to FIGS. 6 and 7 and is intended to receive bone cement exiting from the three-way valve 102. As a result, contamination of the surroundings—i.e. in particular of the surgical area—by bone cement dough is prevented. The hose 104 is connected in pressure-tight manner to the valve system by a sleeve 112 made of metal via a crimping connector.

A static mixer 114 that extends all the way up to the three-way valve 102 is situated in the inside of the tube 103. The static mixer 114 is used to mix the starting components of the bone cement and/or the pre-mixed bone cement dough, when these are pressed through the static mixer 114 in the tube 103.

The rotatable three-way valve 102 is sectioned in the plane of symmetry of the channels seen therein in the cross-sectional views according to FIGS. 11 and 12. Accordingly, the channels are cylindrical and continue in the cut-off part of the three-way valve 102 in mirror-symmetrical manner. The channels form a T-piece in the three-way valve 102. The three-way valve 102 sits in a fitting valve seat 116 that touches tightly against the three-way valve 102 and thus seals the channels, when these are rotated in the valve seat 116. The valve seat 116 has two passages 119 situated in it by which the larger through-going channel in the three-way valve 102 can be connected in fluid-tight manner to the tube 103 on one side and to an insert 118 made of metal for attachment of the hose 104 on the other side.

A draining channel 120 connecting the valve seat 116 to the inside of the collecting container 109 that is closed towards the outside is situated perpendicular to the axis of the two passages 119. The valve seat 116 and the tube 103 are provided as a single part made of plastic material. In the open position of the three-way valve 102 (FIG. 11 bottom, FIG. 12 top), the large through-going channel is connected to the two passages 119 and the small perpendicular channel in the three-way valve 102 is closed through the valve seat 116. Accordingly, the bone cement dough from the cartridge can flow from the tube 103 through the three-way valve 102 and the insert 118 into the hose 104. In the closed position of the three-way valve 102 (FIG. 11 top and FIG. 12 bottom), one side of the large through-going channel is connected to the draining channel 120 to the internal space of the collecting container 109 and the smaller perpendicular channel is connected to the passage 119 to the hose 104, whereas the other passage 119 to the tube 103 is closed by the three-way valve 102. Accordingly, the bone cement dough can flow out of the hose 104 and, if applicable, out of a Luer system adapter (not shown) connected to the hose 104 and/or a connected trocar (not shown) into the collecting container 109. The pressure for this purpose results from an elastic deformation of the hose 104 and, if applicable, trocar that has built up during the extrusion and/or while the bone cement dough was pressed through.

The three-way valve 102 can be rotated manually in the valve seat 116 by a control element (not shown), such as, for example, a T-handle (see the preceding exemplary embodiment). Being cylindrical on the outside, the three-way valve 102 is guided through a cylindrical borehole in the valve seat 116 and is connected to a stopper (not shown in FIGS. 11 and 12, but provided analogous to the preceding exemplary embodiment) on the side opposite from the control element (not shown) and thus is secured against dropping out or against being inadvertently pulled out of the valve seat 116.

Due to the design according to the invention, it is feasible to rapidly interrupt the flow of bone cement dough by rotating and thus closing the three-way valve 102 without large amounts of the bone cement dough continuing to flow through an application opening (not shown), into which the hose 104 or the trocar merge. Simultaneously, leakage of the bone cement dough and thus contamination of the surroundings or user is prevented by the collecting container 109 that takes up any excess of bone cement dough. Moreover, the pressure in the rear side of the bone cement applicator, i.e. between the three-way valve 102 and the dispensing plunger of the cartridge, is maintained such that the flow of bone cement dough can be provided again rapidly after the three-way valve 102 is opened again without the pressure having to be built up again on the rear side of the storage and mixing device.

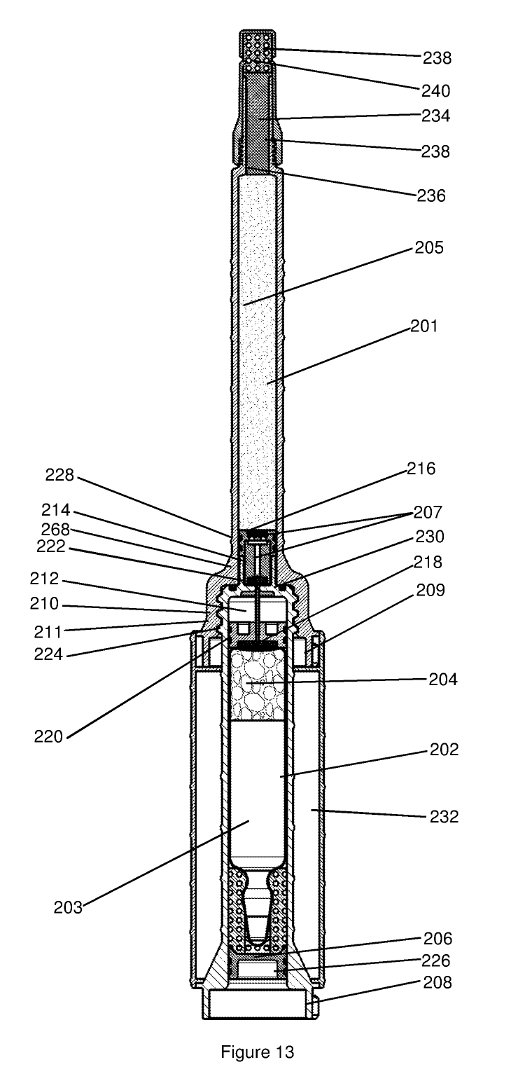

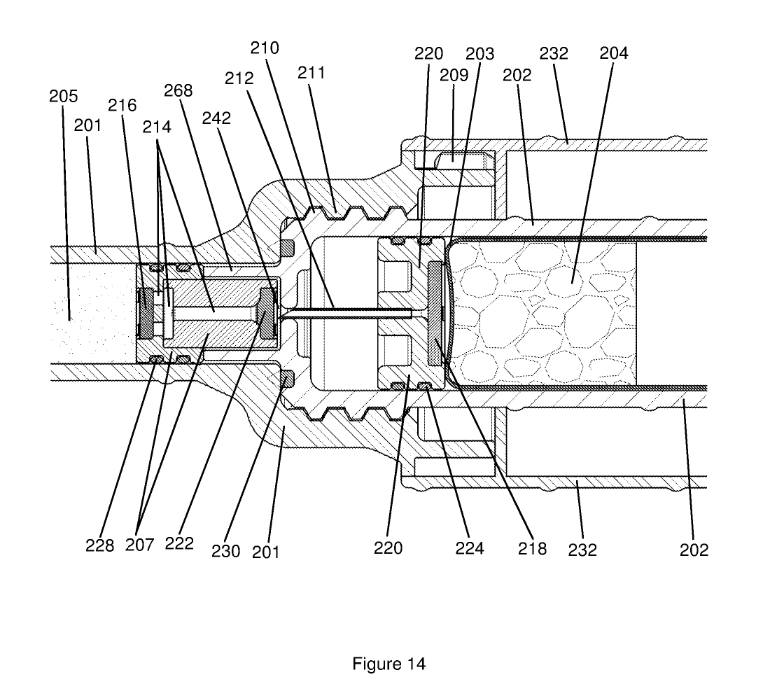

FIGS. 13 and 14 show two schematic cross-sectional views of a third alternative storage and mixing device according to the invention. In this context, FIG. 13 shows a schematic cross-sectional view through the third exemplary embodiment according to the invention and FIG. 14 shows a detail magnification of the connection of the two parts of the third exemplary embodiment according to FIG. 13 before start of the extrusion process as a detail view.

The design of the third alternative storage and mixing device according to the invention is the same as that of the first exemplary embodiment according to FIGS. 1 to 10 unless described otherwise or visualized in FIGS. 13 and 14.

The storage and mixing device has, as front part (on the top in FIG. 13 and on the left in FIG. 14), a cartridge 201 made of plastics that has a cylindrical internal space. The cartridge 201 is detachably connected to a receptacle 202 made of plastics for a glass ampoule 203 (or plastic ampoule 203). The receptacle 202 also comprises a cylindrical internal space into which the glass ampoule 203 is plugged. The monomer liquid 204 is situated in the glass ampoule 203. A cement powder 205 is filled or, preferably, pressed into the internal space of the cartridge 201. The monomer liquid 204 and the cement powder 205 are the starting components 204, 205 of a PMMA bone cement that can be produced with the storage and mixing device. Due to the glass ampoule 203, the monomer liquid 204 can be stored in the receptacle 202 and therefore in the storage and mixing device for very long times.

A feed plunger 206 made of plastics that is mobile in longitudinal direction in the cylindrical internal space of the receptacle 202 is arranged in the receptacle 202. The feed plunger 206 is arranged in the area of the rear side of the receptacle 202. The glass ampoule 203 can be compressed and thereby cracked in the receptacle 202 by the feed plunger 206 by pushing the feed plunger 206 in the direction of the front side, i.e. in the direction of the cartridge 201. The feed plunger 206 comprises, on its front side, wipers by which may assist in wiping fragments of the glass ampoule 203 off the internal wall of the receptacle 202.

A dispensing plunger 207 made of plastics is arranged in the rear side (toward the bottom in FIG. 13 and toward the right in FIG. 14) of the internal space of the cartridge 201. A securing element 208 is provided on the rear side of the receptacle 202 and can be used to connect the receptacle 202 to an extrusion device (not shown in FIGS. 13 and 14). A securing element 209 of identical design is provided on the rear side of the cartridge 201. Both the receptacle 202 and the cartridge 201 can therefore be secured to the same extrusion device. The securing element 208, 209 are preferably suitable and provided for the formation of a bayonet lock. As a result, the feed plunger 206, which is freely accessible from the rear side of the receptacle 202, can be driven forward in the direction of the front side using the same extrusion device as the dispensing plunger 207, which is freely accessible from the rear side of the cartridge 201, when the cartridge 201 is not connected to the receptacle 202.

The cartridge 201 and the receptacle 202 are or can be detachably connected to each other via an external thread 210 on the front side of the receptacle 202 and an internal thread 211 on the rear side of the cartridge 201. The receptacle 202 and the cartridge 201 can be connected to each other in liquid-permeable manner with respect to the monomer liquid 204 by a hollow needle 212 made of a metal or a feedthrough 214 in the dispensing plunger 207. The feedthrough 214 through the dispensing plunger 207 merges through a pore filter 216, which is impermeable to the cement powder 205, but permeable to the monomer liquid 204, into the internal space of the cartridge 201. The feedthrough 214 branches into multiple arms before it enters the internal space of the cartridge 201.

A filter 218, by which the fragments of the glass ampoule 203 can be retained, is arranged in the connection to the hollow needle 212 in the receptacle 202. Instead of the filter 218 or in addition to the filter 218, a screen 218 can be provided just as well or also. The filter 218 is arranged in a connecting plunger 220 that is arranged such as to be mobile in the internal space of the receptacle 202 and has the hollow needle 212 secured to it. In this context, the hollow needle 212 forms the passage through the connecting plunger 220. The connecting plunger 220 is situated at a distance from the front side of the receptacle 202 in this embodiment. In this embodiment, the connection between the internal space of the receptacle 202 and the internal space of the cartridge 201 is generated only upon a motion of the connecting plunger 220.

During a motion of the connecting plunger 220, the hollow needle 212 punctures an elastic membrane 242 (see FIG. 14). When the hollow needle 212 is pulled out of the membrane 242, the membrane 242 seal the dispensing plunger 207, and thus the cartridge 201, on the rear side in liquid-tight manner. Such membranes 242 are known from containers used for drawing up syringes in the field of medicine.

The cylindrical connecting plunger 220 has an external circumference that matches the cylindrical geometry of the internal space of the receptacle 202 and is sealed by two circumferential seals 224 in liquid-tight manner with respect to the internal wall of the receptacle 202. Likewise, the feed plunger 206 is sealed in liquid-tight manner with respect to the internal wall of the receptacle 202 by two circumferential seals 226 and the dispensing plunger 207 is sealed in liquid-tight manner with respect to the internal wall of the cartridge 201 by two circumferential seals 228. Moreover, the cartridge 201 and the receptacle 202 are sealed with respect to each other by a seal 230 that is secured to the front surface of the receptacle 202, when the cartridge 201 and the receptacle 202 are secured to each other. The purpose of all of said seals 224, 226, 228, 230 is to prevent monomer liquid 204 or bone cement from exiting in order to prevent contamination of the surroundings (the OR theatre and the user). For this purpose, the seals 224, 226, 228, 230 can consist of rubber.

In addition, the receptacle 202 is surrounded by a housing 232 made of plastics, whereby the housing 232 is connected to the receptacle 202 and covers, on the front side, the securing element 209 of the cartridge 201, when the cartridge 201 is connected to the receptacle 202. The cartridge 201, the receptacle 202, and the housing 232 can be produced through injection molding.

The front side of the cartridge 201 merges into a dispensing tube 234 that comprises an external thread. A pore filter 236 that is impermeable to the cement powder 205, but is permeable to gases is arranged in the inside of the dispensing tube 234. A cap 238 is secured to the external thread of the dispensing tube 234, whereby the front part of the cap 238 is filled with a Styrofoam or foam 240. The cap 238 can be unscrewed from the dispensing tube 234. The cap 238 comprises lateral openings. Due to this design, the inside of the cartridge 201 and the cement powder 205 can be sterilized with the aid of ethylene oxide, since the openings in the cap 238, the Styrofoam or foam 240, the pore filter 236, and the intervening spaces between the powder particles of the cement powder 205 are permeable to air. Concurrently, air can be pressed out of the receptacle through the cement powder 205, the pore filter 236, the Styrofoam or foam 240, and the openings in the cap 238, when the feed plunger 206 is pressed in the direction of the receptacle 201.

The cement powder 205 is enclosed in the cartridge 201, since all openings are closed by the pore filters 216, 236 such as to be impermeable to the cement powder 205. The content of the cartridge 201 can be sterilized by evacuation and rinsing with ethylene oxide in this context. As a result, the storage and mixing device is also well-suited for long-term storage of the cement powder 205.

The workflow of a method according to the invention is discussed in the following on the basis of the third exemplary embodiment. At the outset of the method, the storage and mixing device is in the starting state that is also shown in FIGS. 13 and 14. Accordingly, the cartridge 201 and the receptacle 202 of the storage and mixing device are initially connected to each other through the threads 210, 211. Being in this state, the storage and mixing device is inserted into an extrusion device in the form of a conventional cartridge gun and is secured to the extrusion device using the securing element 208.

The extrusion device comprises a pestle that can be propelled linearly and is designed analogous to the extrusion device 43 according to FIGS. 1 to 10. The extrusion device 43 is connected to the rear side of the receptacle 202 through an opposite securing element such that the pestle pushes onto the feed plunger 206 and propels it in the direction of the cartridge 201, when the pestle is propelled forward. For this purpose, the pestle is supported such as to be linearly mobile with respect to the opposite securing element and therefore with respect to the receptacle 202.

Operating the extrusion device propels the pestle and, the pestle propels the feed plunger 206 in the direction of the cartridge 201. Since the glass ampoule 203 or plastic ampoule 203 touches against the front side on the connecting plunger 220, the connecting plunger 220 is pushed in the direction of the cartridge 201. The hollow needle 212 punctures the membrane 242 and a plug 222. The purpose of the plug 222 is to stabilize the hollow needle 212. This establishes a liquid-conducting connection between the internal space of the cartridge 201 and the internal space of the receptacle 202. Due to the motion of the feed plunger 206, the feed plunger 206 closes ventilation openings (not shown) that are arranged in the wall of the receptacle 202 in the area of the feed plunger 206 and allow the internal space of the receptacle 202 to be evacuated and sterilized with ethylene oxide in the starting state. The connecting plunger 220 is pushed against the front side of the internal space of the receptacle 202 and cannot be moved any further. As a result, the internal space of the receptacle 202 decreases in size when the feed plunger 206 is propelled further, and the glass ampoule 203 fractures and the monomer liquid 204 exits from the glass ampoule 203 into the internal space of the receptacle 202. Supernatant air from the receptacle 202 is pushed through the filter 218, the feedthrough 214, the pore filter 216, through the intervening spaces of the cement powder 205, through the pore filter 236, through the foam 240, and out of the openings in the cap 238 out of the storage and mixing device.

Lastly, only small fragments of the glass ampoule 203 remain and are retained by the filter 218 and remain in the receptacle 202. The monomer liquid 204 is pressed through the filter 218, the feedthrough 214, and the pore filter 216 into the cement powder 205 and there starts to react with the cement powder 205 such that the bone cement dough is produced from the mixture. At this stage, the storage and mixing device is taken out of the extrusion device. The receptacle 202 and the housing 232 are unscrewed from the cartridge 201. In addition, the cap 238 with the pore filter 238 and the foam 240 is unscrewed and an extended dispensing opening is screwed onto the dispensing tube 234. Subsequently, the cartridge 201 with the extended dispensing opening is secured again to the extrusion device. Earlier, the extrusion device was reset, i.e. the pestle was pushed back again and transitioned into the starting position. After the hollow needle 212 is pulled out, the membrane 242 closes by its own action such that no monomer liquid 204 still present in the feedthrough 214 can exit on the rear side of the cartridge 201, when the cartridge 201 is separate from the receptacle 202.

To secure the extrusion device to the cartridge 201, the opposite securing element of the extrusion device engage the securing element 209 on the rear side of the cartridge 201, which were also used for securing to the securing element 208 of the receptacle 202. Since the securing element 209 on the cartridge 201 and the securing element 208 on the receptacle 202 are identical, both can be secured to the same opposite securing element of the same extrusion device. This simplifies the design and enables the use of conventional extrusion devices. In this context, the securing element 208, 209 and the opposite securing element preferably form a bayonet lock.

The extended dispensing opening can preferably comprise a three-way valve analogous to the first or second embodiment. Likewise, a hose and/or a trocar can be connected to the dispensing tube 234.

The unscrewed rear-side part of the storage and mixing device, namely the receptacle 202 with the fragments 203 of the glass ampoule 203 in it and the housing 232, can be disposed of once they are unscrewed. The front side of the receptacle has a protective tube 268 provided on it, in which the hollow needle 212 is arranged such that the user cannot get injured as easily on the hollow needle 212.

The bone cement dough can be dispensed through the hose and the trocar or through the dispensing tube 234. For this purpose, the dispensing plunger 207 is propelled in the direction of the dispensing tube 234 by the pestle. The bone cement dough from the inside of the cartridge 201 is expelled either directly through the dispensing tube 234 or, if the three-way valve is open, through the dispensing tube 234, through the three-way valve, through the hose, and the trocar and can there be applied to the vertebrae of a patient or theoretically be used for further processing. The three-way valve can be operated, if applicable, in order to interrupt the flow of the bone cement dough.

The features of the invention disclosed in the preceding description and in the claims, figures, and exemplary embodiments, can be essential for the implementation of the various embodiments of the invention both alone and in any combination.

Although illustrated and described above with reference to certain specific embodiments, the present invention is nevertheless not intended to be limited to the details shown. Rather, various modifications may be made in the details within the scope and range of equivalents of the claims and without departing from the spirit of the invention. It is expressly intended, for example, that all ranges broadly recited in this document include within their scope all narrower ranges which fall within the broader ranges.

1. A storage and mixing device for the production of a bone cement dough from a monomer liquid and a cement powder as starting components of the bone cement, the storage and mixing device comprising:
a receptacle, having a monomer liquid container containing the monomer liquid,
a cartridge containing the cement powder,
a feed plunger contacting in the receptacle, whereby the feed plunger is movable in a longitudinal direction of the receptacle and is accessible from a rear side of the receptacle, and
a dispensing plunger in the cartridge, whereby the dispensing plunger is movable in a longitudinal direction within the cartridge,
whereby: a front side of the receptacle is connected or connectable to a rear side of the cartridge such that an internal space of the receptacle is in fluid communication with an internal space of the cartridge such as to be permeable to the monomer liquid, the connection is detachable, and a first securing element is provided on the rear side of the receptacle and a second securing element is provided on the rear side of the cartridge, whereby the first securing element and the second securing element are identical or congeneric.

2. The storage and mixing device according to claim 1, wherein, the dispensing plunger includes:
a closable feedthrough: (1) in fluid communication with an internal space of the cartridge, and (2) including a valve, at least two sealing lips, or a self-closing puncturable membrane,
whereby, the closable feedthrough is adapted to be closable in liquid tight manner upon the connection between the receptacle and the cartridge being detached.

3. The storage and mixing device according to claim 2, wherein the front side of the receptacle includes:
a hollow needle or a tube adapted to connect the inside of the receptacle to the internal space of the cartridge,
whereby, the hollow needle or the tube, together with the closable feedthrough, form a liquid permeable connection into the internal space of the cartridge when the receptacle is connected to the cartridge.

4. The storage and mixing device according to claim 2, wherein the front side of the receptacle includes:
a connecting plunger,
whereby: (1) the connecting plunger is movable in the longitudinal direction within the receptacle, (2) whereby the monomer liquid container is arranged between the connecting plunger and the feed plunger, (3) a passage that is permeable to the monomer liquid and gases is provided in the connecting plunger and the passage is in fluid communication with the internal space of the cartridge, and (4) a motion of the connecting plunger in the direction of the cartridge that is connected to the front side of the receptacle causes the closable feedthrough on the rear side of the cartridge.

5. The storage and mixing device according to claim 1 wherein the front side of the receptacle includes:
a screen or a porous disk that is permeable to gases and liquids and is in fluid communication with the cartridge.

6. The storage and mixing device according to claim 1 wherein,
the storage and mixing device is a bone cement applicator for storage and mixing of the starting components and for application of the bone cement dough in the area of the spine,
whereby the bone cement applicator further comprises an extended dispensing opening connected to the front side of the cartridge, and the extended dispensing opening includes a dispensing tube, a hose, a trocar, or a combination thereof.

7. The storage and mixing device according to claim 6, further comprising:
a three-way valve that is: (1) adapted to be moves between a first position and a second position, (2) operable from outside, and (3) is located in the extended dispensing opening or between the extended dispensing opening and the cartridge,
a collecting container, for reception of bone cement dough, in fluid communication with the three-way valve,
whereby, the extended dispensing opening merges into an application opening at the end of the extended dispensing opening opposite the cartridge, and
whereby, when the three-way valve in the first position, the three-way valve opens a fluid passage between the application opening and the cartridge and closes a discharge channel extending toward the collecting container, and when the three-way valve is in the second position, the three-way valve opens the discharge channel and closes the fluid passage.

8. The storage and mixing device according to claim 1 wherein the receptacle further comprises:
at least one hollow needle or at least one tube on the front side of the receptacle
whereby the receptacle is connected or connectable to the internal space of the cartridge through the needle or tube, and the receptacle is adapted to be liquid-tight except for the needle or tube.

9. The storage and mixing device according to claim 1, wherein,
the monomer liquid container is a glass ampoule, a plastic ampoule, a plastic film bag, or an aluminium-plastic compound bag which is openable in the inside of the receptacle.

10. The storage and mixing device according to claim 1 wherein,
the first securing element and the second securing element is securable to a single holder of a cartridge gun.

11. The storage and mixing device according to claim 1 wherein,
the cross-section of the internal space of the cartridge is at most 4 cm².

12. The storage and mixing device according to claim 1 wherein,
the cartridge is closed on the front side by a cartridge head, an outlet opening is situated in the cartridge head, and the outlet opening is closed by a closure which is impermeable to the cement powder in the cartridge and which is permeable to gas,
whereby the closure is openable through an axial pressure load or by action of a manual force.

13. The storage and mixing device according to claim 1 wherein,
the receptacle is closed or closable on the rear side by the feed plunger in liquid-tight manner.

14. The storage and mixing device according to claim 1 wherein,
the cartridge is hollow cylinder-shaped and the second securing element is adapted to be connected to an extrusion device,
whereby: (1) a cartridge head closes the front side of the hollow cylinder-shaped cartridge, (2) the cartridge head includes an outlet opening, and (3) the outlet opening connects the outside of the cartridge head to the inside of the cartridge head in gas-permeable manner, whereby a dispensing tube is connected to the outlet opening on the cartridge head, and a puncturable membrane is adapted to close the rear side of the dispensing plunger in a liquid-impermeable manner, and
the receptacle is hollow cylinder-shaped,
whereby, the connecting plunger includes: (1) a porous disk, which is permeable to gases and liquids, and (2) at least one hollow needle on the front side of a connecting plunger connected to the rear side of the porous disk by a channel.

15. The storage and mixing device according to claim 1 wherein,
the volume of the monomer liquid in the monomer liquid container is at least as large as the volume of the air-filled intervening spaces between the cement powder particles in the cartridge.

16. The storage and mixing device according to claim 1 wherein,
a wall of the receptacle includes at least one ventilation opening,
whereby: (1) the ventilation opening connects the internal space of the receptacle, in which the monomer liquid container is arranged, to the surroundings of the storing and mixing device, and (2) the ventilation opening is adapted to be closed by a motion of the feed plunger in the direction of the front side of the receptacle before the monomer liquid container is opened through the motion of the feed plunger.

17. The storage and mixing device according to claim 1 wherein,
the cartridge has a compressive strength of more than 10 bar.

18. A method for the production of a bone cement dough from a cement powder and a monomer liquid using a storage and mixing device having a receptacle containing a monomer liquid container with the monomer liquid in it and a cartridge containing the cement powder, whereby the receptacle is secured or securable to the rear side of the cartridge, the method comprising:
a) inserting the storage and mixing device in an extrusion device, having a pestle that is propellable in axial direction;
b) propelling a feed plunger, supported such as to be mobile in the receptacle on the rear side thereof, in the direction of the cartridge by the pestle, whereby: (1) the motion of the feed plunger opens the monomer liquid container and presses the monomer liquid from the monomer liquid container into the cartridge, and (2) the cement powder mixes with the monomer liquid in the internal space of the cartridge;
c) separating the receptacle from the cartridge, whereby the cartridge remains in the extrusion device or is reinserted into the extrusion device after the separation;
d) propelling a dispensing plunger, supported in the cartridge on the rear side thereof such as to be mobile, in the direction of the front side of the cartridge by the pestle, whereby the motion of the dispensing plunger expels the mixture consisting of the cement powder and the monomer liquid from the cartridge in the form of the bone cement dough.

19. The method according to claim 18, wherein
the method is implemented using a storage and mixing device according to claim 1.

20. The method according to claim 18, wherein
the rear side of the cartridge includes a closable feedthrough through which the monomer liquid is pressed into the inside of the cartridge in step b),
whereby, the closable feedthrough is being-closed when the receptacle is separated from the cartridge in step c) or closes self-actingly during the separation.

21. The method according to claim 18 wherein,
a first securing element is provided on the rear side of the receptacle and a second securing element, is provided on the rear side of the cartridge,
whereby, during the insertion of the storage and mixing device in step a), the storage and mixing device is secured to a holder of the extrusion device by the first securing element, and the cartridge is secured to the holder of the extrusion device by the second securing element in step c).

22. The method according to claim 18 wherein
a connecting plunger is arranged on the front side of the receptacle,
whereby: (1) the monomer liquid container is arranged between the connecting plunger and the feed plunger, (2) the connecting plunger is driven in the direction of the cartridge in step b), while the feed plunger is propelled and before the monomer liquid is pressed into the cartridge, (3) a passage into the cartridge is opened such that the internal space of the cartridge and the internal space of the receptacle are connected to each other between the feed plunger and the connecting plunger in liquid-permeable manner, and (4) a crushing force is applied to the liquid container through a motion of the feed plunger against the connecting plunger (20, 220) such that the liquid container is crushed and the monomer liquid is available in the internal space of the receptacle to be pressed into the cartridge.

23. The method according to claim 18 wherein
the mixture of the cement powder and monomer liquid in step d) is pressed out of the cartridge and through an extended dispensing opening, a dispensing tube, a hose, a trocar, a static mixer, or a combination thereof,
whereby the bone cement dough is produced in the cartridge, the extended dispensing opening, the dispensing tube,
the hose, the trocar, or the static mixer.

24. The method according to claim 18 wherein,
a three-way valve, that is: (1) adapted to be moves between a first position and a second position, (2) operable from outside, and (3) located in the extended dispensing opening or downstream from the cartridge,
whereby when the three-way valve is brought into a first position or is in the first position, the three-way valve, provides a continuous connection from the internal space of the cartridge through the extended dispensing opening, and an extrusion of the cartridge by the extrusion device takes place in step d), whereby the bone cement dough mixed from the cement powder and the monomer liquid is pressed through the three-way valve and through the extended dispensing opening, and a subsequent step e) takes place, in which the three-way valve is transitioned into a second position, whereby the three-way valve, being in the second position, stops the flow from the cartridge through the three-way valve and a part of the pressurized bone cement dough downstream from the three-way valve in the extended dispensing opening is pressed through the three-way valve into a collecting container.

25. The method according to claim 24, wherein
the three-way valve is moved to the first position again in a step f) after step e) causing the bone cement dough to be guided again through the three-way valve through the dispensing opening.
